(19) United States
(12) Patent Application Publication
(10) Pub. No.: US 2013/0310262 A1
(43) Pub. Date: Nov. 21, 2013

(54) NONINVASIVE DETECTION OF ROBERTSONIAN TRANSLOCATIONS

(71) Applicant: Ariosa Diagnostics, Inc., San Jose, CA (US)

(72) Inventors: Jacob Zahn, San Jose, CA (US); Arnold Oliphant, San Jose, CA (US); Eric Wang, San Jose, CA (US); Craig Struble, San Jose, CA (US)

(73) Assignee: Ariosa Diagnostics, Inc., San Jose, CA (US)

(21) Appl. No.: 13/899,410

(22) Filed: May 21, 2013

Related U.S. Application Data
(60) Provisional application No. 61/649,738, filed on May 21, 2012.

(51) Int. Cl. C12Q 1/68

(52) U.S. Cl. CPC C12Q 1/6883
USPC 506/2; 435/6.11; 506/9

(57) ABSTRACT

The present invention provides methods for detection of Robertsonian translocations.

### CROSS-REFERENCE TO RELATED APPLICATIONS

This application claims the benefit of provisional Patent Application Ser. No. 61/649,738, filed May 21, 2012 and is incorporated herein by reference.

### FIELD OF THE INVENTION

This invention relates to methods for noninvasive detection of Robertsonian translocations.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Chromosome translocations play significant roles in human fertility, birth defects and cancer. The most common translocations in humans are whole arm exchanges between acrocentric chromosomes (chromosomes 13, 14, 15, 21 and 22), termed Robertsonian (or whole-arm or centric-fusion) translocations, which have an incidence of approximately 1/1000 individuals. When a translocation is balanced, a person with this genetic makeup has 45 rather than 46 chromosomes and is a Robertsonian translocation carrier. Carriers are healthy, have a normal lifespan and may never discover the unusual chromosome arrangement they are carrying.

A person with an unbalanced Robertsonian translocation, however, is generally trisomic for a portion of one of the acrocentric chromosomes. Often fetuses displaying an unbalanced Robertsonian translocation are miscarried in early pregnancy. If a fetus is carried to term, however, a Robertsonian translocation leading to a trisomy may result in, e.g., Down syndrome (the result of an extra chromosome 21), Patau syndrome (the result of an extra chromosome 13), Prader-Willi or Angelman syndrome (the result of an extra portion of chromosome 15), or syndromes of multiple mental and physical developmental disorders. Prenatal screening can identify carriers and potentially fetuses with Robertsonian translocations.

Given the potential biological consequences of passing on an unbalanced Robertsonian translocation; thus, there is a need for methods of screening for such genetic abnormalities. The present invention addresses this need.

### SUMMARY OF THE INVENTION

This Summary is provided to introduce a selection of concepts in simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In one aspect, the methods utilize multiplexed amplification and detection of the sequences of selected nucleic acid regions to calculate a likelihood of the presence or absence of a Robertsonian translocation in one or more individuals. Relative quantities of the selected nucleic acid regions are determined for genomic regions of interest (e.g., an acrocentric chromosome or a portion thereof) using analytical methods as described herein. The analytically determined quantities for the selected nucleic acid regions are then compared to relative quantities of other selected nucleic acid regions and/or one or more reference genomic regions. Such methods are used to detect Robertsonian translocations in DNA isolated from a human patient, preferably a female human patient, and optionally a pregnant human patient. Thus, the methods as described can determine carrier status of a Robertsonian translocation and optionally the presence or absence of a Robertsonian translocation in a patient and/or a fetus.

Thus, in one embodiment, the invention provides a method for detecting the presence or absence of a Robertsonian translocation in an individual comprising the steps of: providing DNA samples from at least five individuals; selectively amplifying one or more selected nucleic acid regions from the p arm of a first chromosome in the samples, wherein the first chromosome is selected from chromosome 13, 14, 15, 21 or 22, and wherein the primers used for selective amplification comprise universal amplification regions; selectively amplifying one or more selected nucleic acid regions from a region outside the p arm of the first chromosome in the samples, wherein the primers used for selective amplification comprise universal amplification regions; further amplifying the selected nucleic acid regions from the at least five individual samples in a single universal amplification reaction; detecting the amplified nucleic acid regions resulting from the single amplification reaction; calculating a relative frequency of the selected nucleic acid regions for an individual sample; comparing the relative frequencies of the selected nucleic acid regions from the p arm of the first chromosome and the selected nucleic acid regions from outside the p arm of the first chromosome for an individual sample; and identifying the presence or absence of a Robertsonian translocation in an individual sample based on the compared relative frequencies.

In some aspects of this embodiment, the DNA samples are from maternal samples. In some aspects, the selected nucleic acid region from the p arm of the first chromosome is a region conserved between acrocentric chromosomes. In some aspects the acrocentric conserved sequence is derived from the sequences of contig NT_{—}167214, FP236241 or AL355134. In some aspects, the selected nucleic acid region from the p arm of the first chromosome is a chromosome-specific genomic region rather than an acrocentric conserved sequence.

In some aspects, a relative frequency of the selected nucleic acid region from the p arm of the first chromosome in an individual that is approximately 20% lower than the relative frequency for the one or more selected nucleic acid regions outside the p arm of the first chromosome in the individual is indicative of a Robertsonian translocation in the individual. In some aspects, a relative frequency of approximately 50% for the selected nucleic acid region from the p arm of the first chromosome of an individual compared to the relative frequency of the one or more selected nucleic acid regions outside the p arm on the first chromosome in that individual is indicative of a Robertsonian translocation.

In some aspects of this embodiment, at least ten selected nucleic acid regions from the p arm of the first chromosome are amplified, and in other aspects, at least twelve, twenty-four, forty-eight, ninety-six or more selected nucleic acid regions from the p arm of the first chromosome are amplified.

In some aspects, the selected nucleic acid regions outside the p arm of the first chromosome is from the q arm of the same chromosome, and in other aspects, the selected nucleic acid regions outside the p arm of the first chromosome are from a second chromosome, and in some aspects both such sequences are used. In some aspects the second chromosome is selected from chromosome 1, 2, 3, 4, 5, 6, 7, 8, 9, 10, 11, 12, 16, 17, 18, 19, 20, or 23. The methods preferably analyze at least ten selected nucleic acid regions for each region outside the p arm of the first chromosome, preferably twenty-four selected nucleic acid regions for each region outside the p arm of the first chromosome, more preferably at least forty-eight selected nucleic acid regions for each region outside the p arm of the first chromosome, and even more preferably at least ninety-six selected nucleic acid regions for each region outside the p arm of the first chromosome.

As described herein, in some aspects of this embodiment, the selected nucleic acid regions are associated with one or more identifying indices, and in some aspects, the frequency of the selected nucleic acid regions is quantified through detection of the associated one or more indices.

In some aspects, the DNA is selectively amplified in a single vessel. In other aspects, the DNA is selectively amplified in different vessels, then pooled.

In some aspects, the selected nucleic acid regions are each counted an average of at least 50, 100, 150, 200, 250, 300 or more times.

In some aspects, there are DNA samples from at least five, ten, twelve or twenty-four individuals, and in other aspects, there are DNA samples from at least forty-eight, seventy-two, ninety-six or more individuals.

In a preferred aspect, the methods utilize detection methods to “count” or quantify the relative frequency of selected nucleic acid regions present in a sample. These frequencies can be utilized to determine if, statistically, the patient and/or the fetus is likely to have a Robertsonian translocation.

The methods of the invention are multiplexed, and preferably highly multiplexed, allowing for multiple selected nucleic acid regions from a single or multiple chromosomes within an individual sample and/or multiple samples to be analyzed simultaneously. In multiplexed methods, the samples can be analyzed separately, or they may be pooled into groups of two or more for analysis of larger numbers of samples. When pooled data is obtained, data is preferably identified for the different samples prior to analysis of the carrier status of a Robertsonian translocation. In some aspects, however, the pooled data may be analyzed for the presence or absence of a Robertsonian translocation and individual samples from the group subsequently analyzed if initial results indicate that a potential aneuploidy is detected within the pooled group.

In certain specific aspects in which the status of the fetus is determined along with status of the mother, the relative percentage of fetal DNA in a maternal sample may be useful for performing or optimizing results obtained from the methods, as the information provides important information on the expected statistical presence of fetal chromosomes and deviation from that expectation may be indicative of fetal aneuploidy due to the presence of the Robertsonian translocation in the fetus.

In yet another aspect, the assay system of the invention can be used to determine if one or more fetus in a multiples pregnancy is likely to have a Robertsonian translocation, and whether further confirmatory tests should be undertaken to confirm the identification of the fetus with the abnormality. For example, the assay system of the invention can be used to determine if one of two twins has a high likelihood of an aneuploidy, followed by a more invasive technique that can distinguish physically between the fetuses, such as amniocentesis or chorionic villi sampling, to determine the identification of the affected fetus.

These and other aspects, features and advantages will be provided in more detail as described herein.

### BRIEF DESCRIPTION OF THE FIGURES

FIG. 1 is a simplified illustration of how Robertsonian translocations result in affected pregnancies.

FIG. 2 is a simplified illustration of an acrocentric chromosome and a chromosome resulting from a Robertsonian translocation and areas from which selected nucleic acid regions may be chosen to identify Robertsonian translocations using the methods of the invention.

FIG. 3 illustrates a multiplexed assay system for detection of two or more selected nucleic acid regions.

FIG. 4 is a graph comparing the frequency of the pTRS63 sequence from the p arm of chromosome 14 from a control sample and in a sample with a known 14-21 Robertsonian translocation.

FIG. 5 is a graph showing the Robertsonian Assay Ratio of samples that were analyzed for acrocentric conserved sequences from the p arm of chromosomes 13, 14, 15, 21 and 22.

### DETAILED DESCRIPTION OF THE INVENTION

The methods described herein may employ, unless otherwise indicated, conventional techniques and descriptions of molecular biology (including recombinant techniques), cell biology, biochemistry, and microarray and sequencing technology, which are within the skill of those who practice in the art. Such conventional techniques include polymer array synthesis, hybridization and ligation of oligonucleotides, sequencing of oligonucleotides, and detection of hybridization using a label. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, L., Biochemistry (4th Ed.) W.H. Freeman, New York (1995); Gait, “Oligonucleotide Synthesis: A Practical Approach” IRL Press, London (1984); Nelson and Cox, Lehninger, Principles of Biochemistry, 3^{rd} Ed., W. H. Freeman Pub., New York (2000); and Berg et al., Biochemistry, 5^{th} Ed., W.H. Freeman Pub., New York (2002), all of which are herein incorporated by reference in their entirety for all purposes. Before the present compositions, research tools and methods are described, it is to be understood that this invention is not limited to the specific methods, compositions, targets and uses described, as such may, of course, vary. It is also to be understood that the terminology used herein is for the purpose of describing particular aspects only and is not intended to limit the scope of the present invention, which will be limited only by the appended claims.

It should be noted that as used herein and in the appended claims, the singular forms “a,” “an,” and “the” include plural referents unless the context clearly dictates otherwise. Thus, for example, reference to “a nucleic acid region” refers to one, more than one, or mixtures of such regions, and reference to “a method” includes reference to equivalent steps and methods known to those skilled in the art, and so forth.

Where a range of values is provided, it is to be understood that each intervening value between the upper and lower limit of that range—and any other stated or intervening value in that stated range—is encompassed within the invention. Where the stated range includes upper and lower limits, ranges excluding either of those limits are also included in the invention.

All publications mentioned herein are incorporated by reference for all purposes including the purpose of describing and disclosing formulations and methodologies that that might be used in connection with the presently described invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “amplified nucleic acid” is any nucleic acid molecule whose amount has been increased at least two fold by any nucleic acid amplification or replication method performed in vitro as compared to its starting amount.

The term “diagnostic tool” as used herein refers to any composition or method of the invention used in, for example, a system in order to carry out a diagnostic test or assay on a patient sample.

The term “hybridization” generally means the reaction by which the pairing of complementary strands of nucleic acid occurs. DNA is usually double-stranded, and when the strands are separated they will re-hybridize under the appropriate conditions. Hybrids can form between DNA-DNA, DNA-RNA or RNA-RNA. They can form between a short strand and a long strand containing a region complementary to the short one. Imperfect hybrids can also form, but the more imperfect they are, the less stable they will be (and the less likely to form).

The term “likelihood” refers to any value achieved by directly calculating likelihood or any value that can be correlated to or otherwise indicate a likelihood.

The terms “locus” and “loci” as used herein refer to a nucleic acid region of known location in a genome.

The term “maternal sample” as used herein refers to any sample taken from a pregnant mammal which comprises both fetal and maternal cell free genomic material (e.g., DNA). Preferably, maternal samples for use in the invention are obtained through relatively non-invasive means, e.g., phlebotomy or other standard techniques for extracting peripheral samples from a subject.

“Microarray” or “array” refers to a solid phase support having a surface, preferably but not exclusively a planar or substantially planar surface, which carries an array of sites containing nucleic acids such that each site of the array comprises substantially identical or identical copies of oligonucleotides or polynucleotides and is spatially defined and not overlapping with other member sites of the array; that is, the sites are spatially discrete. The array or microarray can also comprise a non-planar interrogatable structure with a surface such as a bead or a well. The oligonucleotides or polynucleotides of the array may be covalently bound to the solid support, or may be non-covalently bound. Conventional microarray technology is reviewed in, e.g., Schena, Ed., Microarrays: A Practical Approach, IRL Press, Oxford (2000). “Array analysis”, “analysis by array” or “analysis by microarray” refers to analysis, such as, e.g., isolation of specific nucleic acids or sequence analysis of one or more biological molecules using a microarray.

By “non-polymorphic”, when used with respect to detection of selected nucleic acid regions, is meant detection of a nucleic acid region, which may contain one or more polymorphisms, but in which the detection is not reliant on detection of the specific polymorphism within the region. Thus a selected nucleic acid region may contain a polymorphism, but detection of the region using the methods of the invention is based on occurrence of the region rather than the presence or absence of a particular polymorphism in that region.

The terms “oligonucleotides” or “oligos” as used herein refer to linear oligomers of natural or modified nucleic acid monomers, including deoxyribonucleotides, ribonucleotides, anomeric forms thereof, peptide nucleic acid monomers (PNAs), locked nucleotide acid monomers (LNA), and the like, or a combination thereof, capable of specifically binding to a single-stranded polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g., 8-12, to several tens of monomeric units, e.g., 100-200 or more.

As used herein the term “polymerase” refers to an enzyme that links individual nucleotides together into a long strand, using another strand as a template. There are two general types of polymerase—DNA polymerases, which synthesize DNA, and RNA polymerases, which synthesize RNA. Within these two classes, there are numerous sub-types of polymerases, depending on what type of nucleic acid can function as template and what type of nucleic acid is formed.

As used herein “polymerase chain reaction” or “PCR” refers to a technique for replicating a specific piece of target DNA in vitro, even in the presence of excess non-specific DNA. Primers are added to the target DNA, where the primers initiate the copying of the target DNA using nucleotides and, typically, Taq polymerase or the like. By cycling the temperature, the target DNA is repetitively denatured and copied. A single copy of the target DNA, even if mixed in with other, random DNA, can be amplified to obtain billions of replicates. The polymerase chain reaction can be used to detect and measure very small amounts of DNA and to create customized pieces of DNA. In some instances, linear amplification methods may be used as an alternative to PCR.

The term “polymorphism” as used herein refers to any genetic changes in a locus that may be indicative of that particular loci, including but not limited to single nucleotide polymorphisms (SNPs), methylation differences, short tandem repeats (STRs), and the like.

Generally, a “primer” is an oligonucleotide used to, e.g., prime DNA extension, ligation and/or synthesis, such as in the synthesis step of the polymerase chain reaction or in the primer extension techniques used in certain sequencing reactions. A primer may also be used in hybridization techniques as a means to provide complementarity of a nucleic acid region to a capture oligonucleoitide for detection of a specific nucleic acid region.

The term “research tool” as used herein refers to any method of the invention used for scientific enquiry, academic or commercial in nature, including the development of pharmaceutical and/or biological therapeutics. The research tools of the invention are not intended to be therapeutic or to be subject to regulatory approval; rather, the research tools of the invention are intended to facilitate research and aid in such development activities, including any activities performed with the intention to produce information to support a regulatory submission.

The term “selected nucleic acid region” as used herein refers to a nucleic acid region corresponding to an individual chromosome. Selected nucleic acid regions may be directly isolated and enriched from the sample for detection, e.g., based on hybridization and/or other sequence-based techniques, or they may be amplified using the sample as a template prior to detection of the sequence.

The terms “selective amplification” and “selectively amplify” and the like refer to an amplification procedure that depends in whole or in part on hybridization of an oligo to a sequence in a selected nucleic acid region. In certain selective amplifications, the primers used for amplification are complementary to a selected nucleic acid region. In other selective amplifications, the primers used for amplification are universal primers, but they only result in a product if a region of the nucleic acid used for amplification is complementary to a selected nucleic acid region of interest.

The terms “sequencing” and “sequence determination” and the like as used herein refer generally to any and all biochemical methods that may be used to determine the order of nucleotide bases in a nucleic acid.

The terms “specifically binds” and “specific binding” and the like as used herein, when referring to a binding partner (e.g., a nucleic acid probe or primer, antibody, etc.) result in the generation of a statistically significant positive signal under the designated assay conditions. Typically the interaction will subsequently result in a detectable signal that is at least twice the standard deviation of any signal generated as a result of undesired interactions (background).

The term “universal” when used to describe an amplification procedure refers to the use of a single primer or set of primers for a plurality of amplification reactions. For example, in the detection of 96 different target sequences, all the templates may share identical universal priming sequences, allowing for the multiplex amplification of the 96 different sequences using a single set of primers. The use of such primers greatly simplifies multiplexing in that only two primers are needed to amplify a plurality of selected nucleic acid sequences. The term “universal” when used to describe a priming site is a site to which a universal primer will hybridize. It should also be noted that “sets” of universal priming sequences/primers may be used. For example, in highly multiplexed reactions, it may be useful to use several sets of universal sequences, rather than a single set; for example, 96 different nucleic acids may have a first set of universal priming sequences, and the second 96 a different set of universal priming sequences, etc.

### The Invention in General

The present invention provides improved methods for identifying copy number variants of particular genomic regions, particularly for chromosomes known to be involved in Robertsonian translocations, in biological samples. The detection methods of the invention are not reliant upon the presence or absence of any polymorphic or mutation information, and thus are agnostic as to the type of genetic variation, if any, that may be present in the selected nucleic acid regions under interrogation. The methods of the invention are useful for, e.g., any sample containing nucleic acids when assessing paternal or maternal carrier status, or any sample containing fetal nucleic acids when assessing the probability of a Robertsonian translocation in a fetus.

The assay methods of the invention include selective enrichment of selected nucleic acid regions from chromosomes of interest and/or reference chromosomes. A distinct advantage of the invention is that the selected nucleic acid regions can be further analyzed using a variety of detection and quantification techniques, including but not limited to hybridization techniques, digital PCR and high-throughput sequencing determination techniques. Probes can be designed against any number of selected nucleic acid regions for any chromosome. Although amplification prior to the identification and quantification of the selected nucleic acid regions is not mandatory, limited amplification prior to detection is preferred.

The present invention is an improvement over more random techniques such as massively parallel, shotgun sequencing, and the use of random digital PCR which have been used to detect copy number variations in maternal samples such as maternal blood. The aforementioned approach relies upon sequencing of all or a statistically significant population of DNA fragments in a sample, followed by mapping of or otherwise associating the fragments to their appropriate chromosomes. The identified fragments are then compared against each other or against some other reference (e.g., a sample with a known normal chromosomal complement) to determine copy number variation of particular chromosomes. Such methods are inherently inefficient as compared to the present invention, as the data generated on the chromosomes of interest (e.g., the selected nucleic acid regions) constitute only a minority of the data that is generated.

Techniques that are dependent upon a very broad sampling of DNA in a sample provide a broad coverage of the DNA analyzed, but in fact are sampling the DNA contained within a sample on a 1× or less basis (i.e., subsampling). In contrast, the selective amplification and/or enrichment techniques (such as hybridization) used in the present methods provide depth of coverage of only the selected nucleic acid regions; and as such provide a “super-sampling” of the selected nucleic acid regions with an average sequence coverage of preferably 2× or more, more preferably sequence coverage of 100× of more, even more preferably sequence coverage of 1000× or more of the selected nucleic acid regions.

Thus, the substantial majority of sequences analyzed for identification of Robertsonian translocations are informative of the presence of one or more selected nucleic acid regions on one or more chromosomes of interest and/or a reference chromosome. The methods of the invention do not require the analysis of large numbers of sequences which are not from the chromosomes of interest and which do not provide information on the relative quantity of the chromosomes of interest.

### Robertsonian Translocations

Robertsonian translocation is a common form of chromosomal rearrangement that in humans occurs in the five acrocentric chromosome pairs, namely 13, 14, 15, 21 and 22. Other translocations do occur but do not lead to a viable fetus. A Robertsonian translocation is a type of nonreciprocal translocation involving two homologous (paired) chromosomes or non-homologous chromosomes (i.e., two different chromosomes, not belonging to a homologous pair). A feature of chromosomes that are commonly found to undergo such translocations is that they possess an acrocentric centromere, partitioning the chromosome into a long arm q arm containing the vast majority of genes, and a short arm p arm with a much smaller proportion of genetic content.

In most Robertsonian translocations, the participating chromosomes break at their centromeres and the long q arms fuse to form a single chromosome. The short p arms also join to form a single chromosome; however, this small chromosome typically contains nonessential genes and is usually lost within a few cell divisions. Thus, the result of a Robertsonian translocation is typically loss of short p arm sequences; however, little genetic material is lost and the individual will be normal with a full complement of essential genetic material despite the translocation. Individuals with Robertsonian translocations have only 45 chromosomes in each of their cells, yet all essential genetic material is present and they appear normal. However, the children of these individuals may either be normal and carry the q-arm fusion chromosome, or they may inherit the q-arm fusion chromosome and two sister acrocentric chromosomes possessing the one of the same q arms as in the q-arm fusion chromosome.

All ten possible pairwise combinations of the five acrocentric chromosomes resulting in non-homologous Robertsonian translocations have been observed, but the distribution of these different types of translocations is highly nonrandom. Robertsonian 13q14q and 14q21q translocations are far more common than the remaining types of Robersonians. In studies using newborn screening and prenatal testing for advanced maternal age, 13q14q and 14q21q comprise 75.6% and 9.9%, respectively, of all non-homologous Robertsonian translocations. Each of the other types make up only 0.8% to 3.7% of the total number of Robertsonian translocations (Page, et al., Human Molecular Genetics, 5(9):1279-88 (1996)). The excess of 14q21q is even more evident among Robertsonian translocations identified through Down syndrome. About one in a thousand newborns has a Robertsonian translocation.

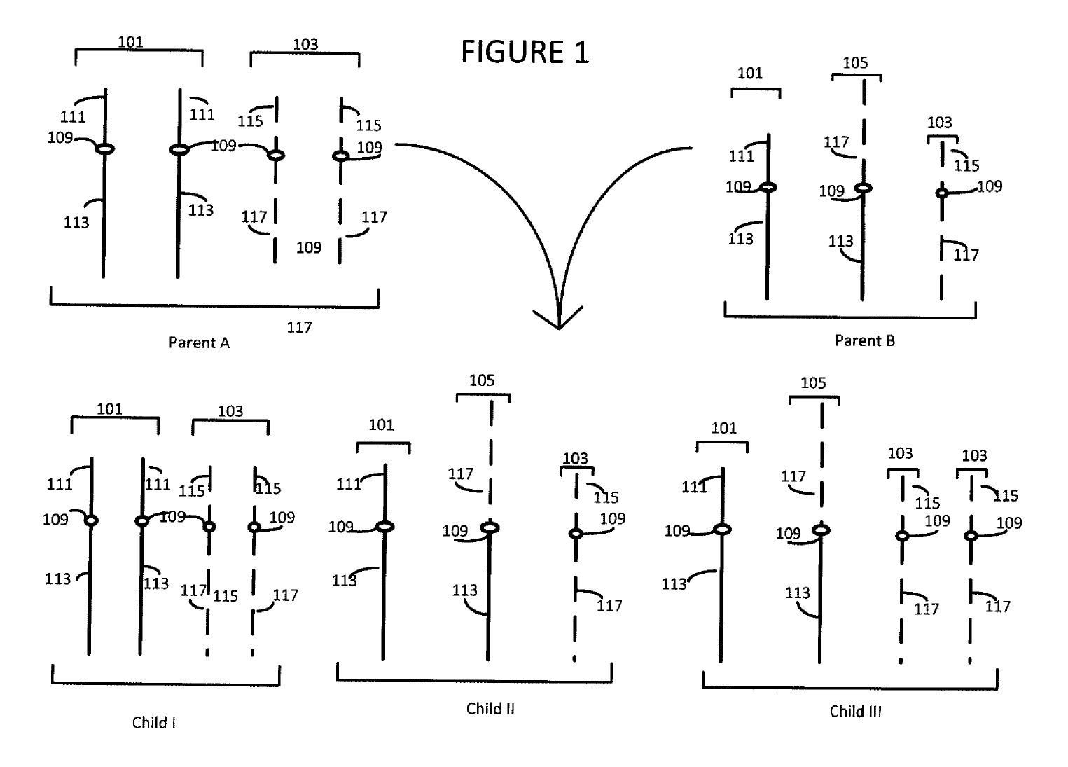

FIG. 1 is a simplified illustration of acrocentric chromosomes involved in a Robertsonian translocation. FIG. 1 shows parents A and B. Parent A has a normal chromosome complement: two copies of a chromosome, e.g., 14 (101) and two copies of chromosome, e.g., 21 (103). Chromosome 14 (101) comprises a long or q arm (113), a centromere (109) and a short or p arm (111), and chromosome 21 (103) comprises a long or q arm (117), a centromere (109) and a short or p arm (115). Parent B has a Robertsonian translocation: one copy of chromosome 14 (101), one copy of chromosome 21 (103), and one q-arm fusion chromosome (105). The normal chromosome 14 (101) comprises a long or q arm (113), a centromere (109) and a short or p arm (111) as in Parent A. The normal chromosome 21 (103) comprises a long or q arm (117), a centromere (109) and a short or p arm (115) as in Parent A. However, Parent B has a q-arm fusion chromosome (105), comprising the long or q arm (113) of chromosome 14, a centromere (109), and the long or q arm (117) of chromosome 21.

The offspring of Parent A and Parent B may be one of three genotypes: Offspring I will be normal, will not inherit the q-arm fusion chromosome, and will have 46 chromosomes with two normal copies of chromosome 14 (101) and two normal copies of chromosome 21 (103). Offspring I will grow and develop normally. Offspring II inherits a normal copy of chromosome 14 (101), a normal copy of chromosome 21 (103), and a q-arm fusion chromosome (105); however, like the carrier parent, Offspring II will grow and develop normally. Offspring III, on the other hand, will have an aneuploidy—a trisomy—due to inheriting one copy of chromosome 14 (101), the q-arm fusion chromosome (105), and two copies of chromosome 21 (103). Offspring III thus has three q arms from chromosome 21, and this particular trisomy leads to Down Syndrome.

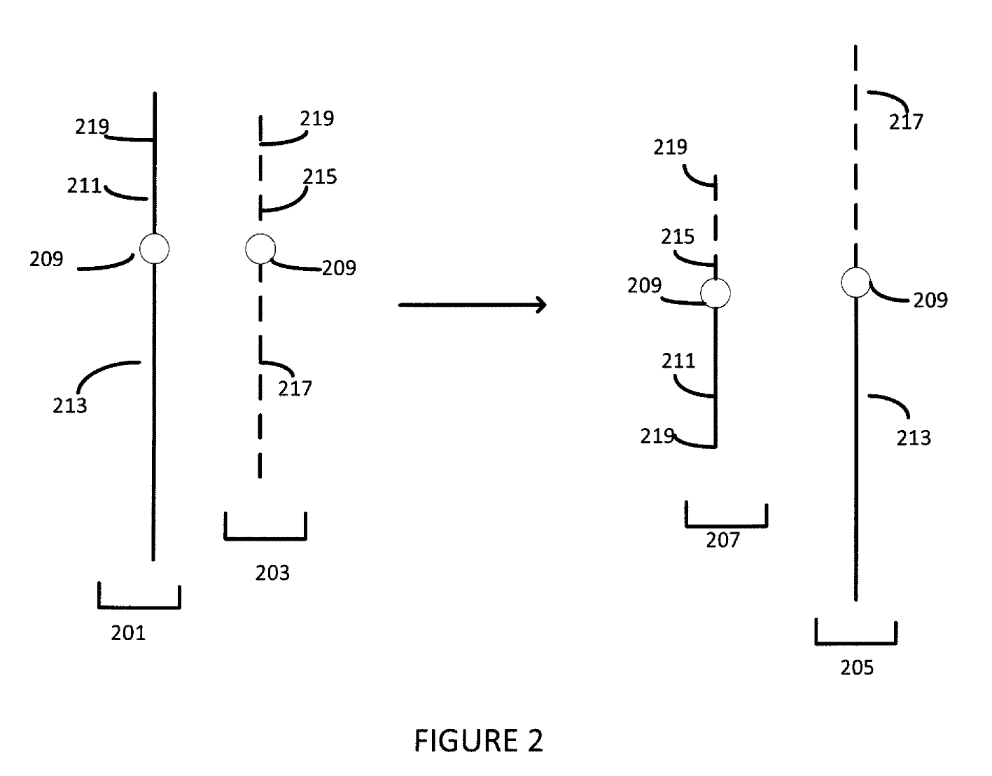

FIG. 2 is a simplified illustration of two acrocentric chromosomes and two chromosomes resulting from a Robertsonian translocation between the two acrocentric chromosomes. Two acrocentric chromosomes are shown, for example, chromosome 14 (201) and chromosome 21 (203). Chromosome 14 (201) comprises a long or q arm (213), a centromere (209) and a short or p arm (211), and chromosome 21 (203) comprises a long or q arm (217), a centromere (209) and a short or p arm (215). Recombination between chromosome 14 (201) and chromosome 21 (203) results in two chromosomes having Robertsonian translocations. The first recombined chromosome is a q-arm fusion chromosome (205), comprising the long or q arm (213) of chromosome 14, a centromere (209), and the long or q arm (217) of chromosome 21. The second recombined chromosome is a p-arm fusion chromosome (207), comprising the short or p arm (211) of chromosome 14, a centromere (209), and the short or p arm (215) of chromosome 21. P-arm fusion chromosomes contain nonessential genes and are usually lost within a few cell divisions and not present in the parent or offspring.

Looking at the q-arm fusion chromosome (205) comprising the long or q arm (213) of chromosome 14, a centromere (209), and the long or q arm (217) of chromosome 21, it is clear that an individual with a Robertsonian translocation—either a carrier or an individual that is trisomic for one of the long or q arms of one of the chromosomes—will lack one or two of the short or p arm sequences, respectively. Looking back at FIG. 1, Parent B lacks two short or p arm sequences, one for each of chromosome 14 and 21. Offspring II, a carrier like Parent B, also lacks two short or p arm sequences, one for each of chromosome 14 and 21. Offspring III, on the other hand, lacks only one short or p arm sequence, here the short or p arm sequence of chromosome 14, but has three long or q arm sequences for chromosome 21. The methods for noninvasive detection of Robertsonian translocations according to the invention exploit these copy number anomalies.

In one embodiment, the methods of the present invention quantify sequences from the short or p arms of acrocentric chromosomes to detect Robertsonian translocations. Chromosomes 14 and 21 as shown in FIG. 2—and indeed all acrocentric chromosomes 13, 14, 15, 21 and 22—comprise conserved sequences, including sequences encoding ribosomal RNA (for example, the human ribosomal DNA repeating unit as found in U13369) and other acrocentric conserved sequences on the end of their p arms distal to the centromere (shown as 219 in FIG. 2). Thus, a normal disomic individual will have ten copies of the acrocentric conserved sequences: there will be one copy on each of the p arms of both copies of each of the five acrocentric chromosomes. In some cases—as with a Robertsonian translocation carrier such as Parent B or Offspring II of FIG. 1—two copies (from the p arms of chromosomes 14 and 21) out of ten acrocentric conserved sequences will be lacking; that is, there will be a 20% decrease in the relative frequency of acrocentric conserved sequences in these individuals. In other cases—as with the trisomic Offspring III from FIG. 1—one copy (from the p arm of chromosome 21) out of ten acrocentric conserved sequences will be lacking; that is, there will be a 10% decrease in the relative frequency of acrocentric conserved sequences in these individuals. Thus, in some embodiments, the methods of the present invention select one or more acrocentric conserved sequences for the selected nucleic acid regions to be amplified, detected and quantified in the methods of the present invention. One acrocentric conserved sequence of particular use is the contig NT_{—}167214 (AL592188) (see, e.g., Cole, et al, Nature, 431(7011):931-945 (2004) and the NCBI database, human DNA sequence from clone RP11-337M7 on chromosome 22). Other acrocentric conserved sequences are FP236241 (human DNA sequence from clone CH507-338C24 on chromosome 21) and AL355124 (human DNA sequence from clone RP11-398A7 on chromosome 13).

In other embodiments, other sequences from the p arms of acrocentric chromosomes (“p arm sequences”) may be used as an alternative to or, preferably, in addition to the acrocentric conserved sequences such as chromosome-specific sequences (that is, sequences on the p arm of an acrocentric chromosome that is specific or unique to that chromosome). Any sequences from the p arms of acrocentric chromosomes may be used; however, sequences that are relatively distal to the centromere and proximal the ribosomal RNA coding sequences are preferred, as recombination between the acrocentric chromosomes appears to take place in the p arm proximal to the centromere (see, e.g., Page, et al., Human Molecular Genetics, 5(9):1279-88 (1996); Earle, et al., Am. J. Hum. Genet., 50:717-24 (1992); and Han, Am. J. Hum. Genet., 55:960-67 (1994)). One particular sequence from the p arm of chromosome 14 that has been identified to be lost in Robertsonian translocations is pTRS-63 (again see Page, et al.). As with the acrocentric conserved sequences, in some cases—as with a Robertsonian translocation carrier such as Parent B or Offspring II of FIG. 1—two copies out of ten p arm sequences will be lacking; that is, there will be a 20% decrease in the relative frequency of p arm sequences in these individuals. In other cases—as with the trisomic Offspring III of FIG. 1—one copy out of ten p arm sequences will be lacking; that is, there will be a 10% decrease in the relative frequency of p arm sequences in these individuals.

In addition to looking at a decrease in frequency in p arm sequences including acrocentric conserved sequences, q arm trisomy may be detected using selected nucleic acid regions from the q arms (“q arm sequences”) of acrocentric chromosomes, and looking at the relative frequencies of these q arm sequences. Looking at the frequencies of q arm sequences in FIG. 1, Parent A, Parent B, Offspring I and Offspring II have the same q arm frequencies for the q arms from chromosomes 14 and 21, and the frequency of these q arms should be relatively the same as the frequency of selected nucleic acid regions from the q or p arms from other, non-acrocentric chromosomes. However, Offspring III will have a “normal” frequency for chromosome 14 (that is the same relative frequency as Parent A, Parent B, Offspring I and Offspring II) but an increased frequency for chromosome 21, indicating a trisomy. The q arm sequences chosen to be selected nucleic acid regions to be amplified, detected and quantified by the methods of the invention may be any sequences that lie on the q arm of the chromosome of choice, though preferably the selected nucleic acid regions selected are sequences unique to the chromosome.

### Amplification Methods

Numerous amplification methods may be used to selectively amplify the selected nucleic acid regions that are analyzed (e.g., sequenced) in the methods of the invention, increasing the copy number of the selected nucleic acid regions in a manner that allows preservation of the relative quantity of the selected nucleic acid regions in the initial sample. Although not all combinations of amplification and analysis are described herein in detail, it is well within the skill of those in the art to utilize different, comparable amplification and/or analysis methods to analyze the selected nucleic acid regions consistent with this specification, as such variations should be apparent to one skilled in the art upon reading the present disclosure.

Amplification methods useful in the present invention include but are not limited to, polymerase chain reaction (PCR) (U.S. Pat. Nos. 4,683,195; and 4,683,202; PCR Technology: Principles and Applications for DNA Amplification, ed. H. A. Erlich, Freeman Press, NY, N.Y., 1992), ligase chain reaction (LCR) (Wu and Wallace, Genomics 4:560, (1989); Landegren et al., Science 241:1077 (1988)), strand displacement amplification (SDA) (U.S. Pat. Nos. 5,270,184; and 5,422,252), transcription-mediated amplification (TMA) (U.S. Pat. No. 5,399,491), linked linear amplification (LLA) (U.S. Pat. No. 6,027,923), and the like, self-sustained sequence replication (Guatelli et al., PNAS USA, 87:1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NASBA) (see, U.S. Pat. Nos. 5,409,818, 5,554,517, and 6,063,603, each of which is incorporated herein by reference). Other amplification methods that may be used include: Qbeta Replicase, described in PCT Patent Application No. PCT/US87/00880, isothermal amplification methods such as SDA, described in Walker et al., Nucleic Acids Res. 20(7):1691-6 (1992), and rolling circle amplification, described in U.S. Pat. No. 5,648,245. Yet other amplification methods that may be used are described in, U.S. Pat. Nos. 5,242,794, 5,494,810, 4,988,617 and in U.S. Ser. No. 09/854,317 and US Pub. No. 20030143599, each of which is incorporated herein by reference. In some aspects DNA is amplified by multiplex locus-specific PCR. In a preferred aspect the DNA is amplified using adaptor-ligation and single primer PCR. Other available methods of amplification include balanced PCR (Makrigiorgos et al., Nat Biotechnol, 20:936-39 (2002)) and self-sustained sequence replication (Guatelli et al., PNAS USA, 87:1874 (1990)). Based on such methodologies, a person skilled in the art can readily design primers in any suitable regions 5′ and 3′ to a selected nucleic acid region of interest. Such primers may be used to amplify DNA of any length so long that it contains the selected nucleic acid region of interest in its sequence.

The length of the selected nucleic acid regions most preferably are long enough to provide enough sequence information to distinguish the selected nucleic acid regions from one another. Generally, a selected nucleic acid region is at least about 16 nucleotides in length, and more typically, a selected nucleic acid region is at least about 20 nucleotides in length. In a preferred aspect of the invention, the selected nucleic acid regions are at least about 30 nucleotides in length. In a more preferred aspect of the invention, the selected nucleic acid regions are at least about 32, 40, 45, 50, or 60 nucleotides in length. In other aspects of the invention, the selected nucleic acid regions can be about 100, 150 or up to 200 in length.

In certain aspects, the selective amplification process uses one or a few rounds of amplification with primer pairs comprising nucleic acids complementary to the selected nucleic acid regions (a sequence-specific amplification process). In other aspects, the selective amplification comprises an initial linear amplification step (also a sequence-specific amplification process). Linear amplification methods can be particularly useful if the starting amount of DNA is quite limited. Linear amplification increases the amount of DNA molecules in a way that is representative of the original DNA content, which helps to reduce sampling error in cases such as the present invention where accurate quantification of the selected nucleic acid regions is needed.

Thus, in preferred aspects, a limited number of cycles of sequence-specific amplification are performed on the starting maternal sample comprising cell free DNA. The number of cycles is generally less than that used for a typical PCR amplification, e.g., 5-30 cycles or fewer.

Primers or probes are designed to amplify the selected nucleic acid regions. The primers for selective amplification are preferably designed to 1) efficiently amplify selected nucleic acid regions from the chromosome of interest; 2) have a predictable range of expression from maternal and/or fetal sources in different maternal samples; and 3) be distinctive to the particular chromosome of interest, i.e., not amplify homologous regions on other chromosomes. The primers or probes may be modified with an end label at the 5′ end (e.g., with biotin) or elsewhere along the primer or probe such that the amplification products can be purified or attached to a solid substrate (e.g., bead or array) for further isolation or analysis. In a preferred aspect, the primers are engineered to have, e.g., compatible melting temperatures, to be used in multiplexed reactions that allow for the amplification of several to many selected nucleic acid regions such that a single reaction yields multiple DNA copies from different selected nucleic acid regions. Amplification products from the linear amplification may then be further amplified with standard PCR methods or with additional linear amplification.

Cell free DNA can be isolated from, e.g., whole blood, plasma, or serum from a pregnant woman, and incubated with primers engineered to amplify a set number of selected nucleic acid regions that correspond to chromosomes of interest. Preferably, the number of primer pairs used for initial amplification (and thus the number of selected nucleic acid regions) will be 12 or more, more preferably 24 or more, more preferably 36 or more, even more preferably 48 or more, and even more preferably 96 or more. Each of the primer pairs corresponds to a single selected nucleic acid region, and the primer pairs are optionally tagged for identification (e.g., by used of indexes) and/or isolation (e.g., comprise a nucleic acid sequence or chemical moiety that is utilized for capture). A limited number of amplification cycles, preferably 10 or fewer, are performed. The amplification products (the amplified selected nucleic acid regions) are subsequently isolated by methods known in the art. For example, when the primers are linked to a biotin molecule, the amplification products can be isolated via binding to avidin or streptavidin on a solid substrate. The amplification products may then be subjected to further biochemical processes such as further amplification with other primers (e.g., universal primers) and/or detection techniques such as sequence determination and hybridization.

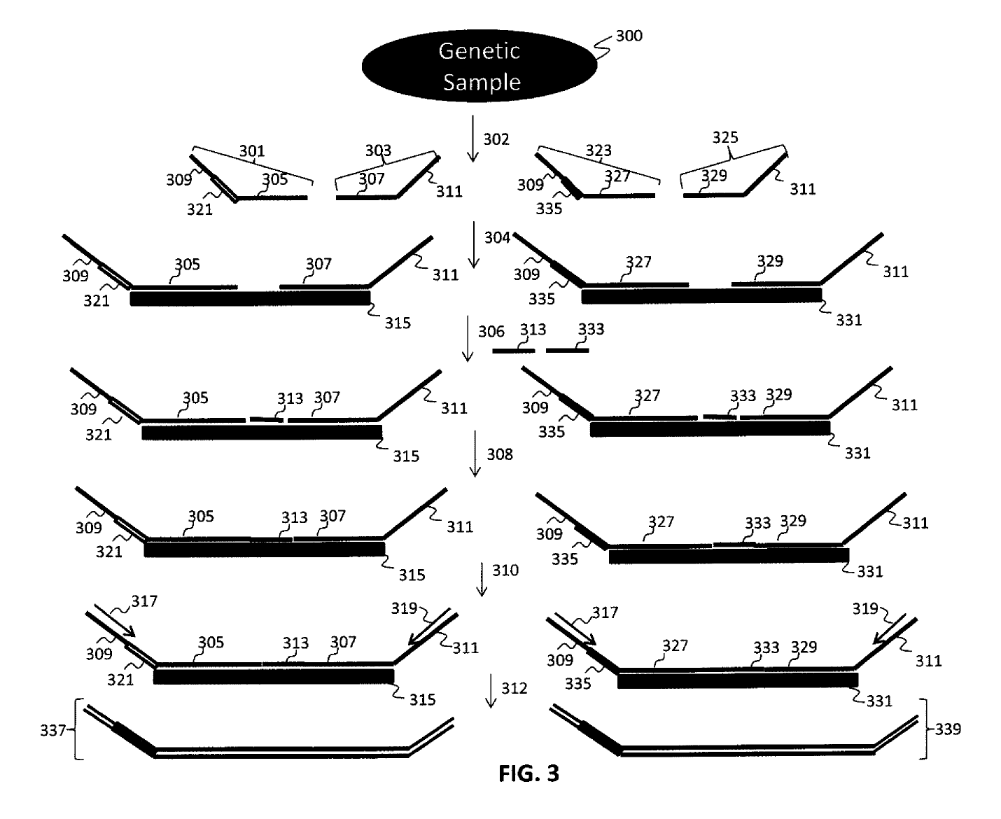

FIG. 3 illustrates one exemplary method embodiment where two different selected nucleic acid regions are detected in a single tandem reaction assay. Such method embodiments, assay systems and related embodiments are described in detail in, e.g., U.S. Ser. Nos. 13/013,732, filed Jan. 25, 2011; 13/245,133, filed Sep. 26, 2011; 13/205,570, filed Aug. 8, 2011; 13/293,419, filed Nov. 10, 2011; 13/205,409, filed Aug. 8, 2011; 13/205,603, filed Aug. 8, 2011; 13/407, 978, filed Feb. 29, 2012; 13/274,309, filed Oct. 15, 2011; 13/316,154, filed Dec. 9, 2011, and 13/338, 963, filed Dec. 28, 2011, all of which are incorporated herein in their entirety. Two sets of fixed sequence oligonucleotides (301 and 303, 323 and 325) that specifically hybridize to two different selected nucleic acid regions 315, 331 are introduced 302 to a genetic sample and allowed to hybridize 304 to the respective selected nucleic acid regions. Each set of fixed sequence oligonucleotides comprises an oligonucleotide 301, 323 having a sequence specific region 305, 327, a universal primer region 309 and an index region 321, 335. The other fixed sequence oligonucleotide in a set comprises a sequence specific region 307, 329 and a universal primer region 311.

Following hybridization, the unhybridized fixed sequence oligonucleotides are preferably separated from the remainder of the sample (not shown). Bridging oligos 313, 333 are introduced to the hybridized pair of fixed sequence oligonucleotide/nucleic acid regions and allowed to hybridize 306 to these regions. Although shown in FIG. 3 as two different bridging oligonucleotides, in fact the same bridging oligo may be suitable for both hybridization events, or they may be two oligonucleotides from a pool of degenerate oligos. The hybridized oligonucleotides are ligated 208 to create a contiguous nucleic acid spanning and complementary to each selected nucleic acid region of interest. Following ligation, universal primers 317, 319 are introduced to amplify 310 the ligated oligonucleotides to create 312 amplification products 337, 339 that comprise the sequence of the selected nucleic acid regions of interest. These amplification products 337, 339 are optionally isolated, detected and/or quantified to provide information on the presence and amount of the selected nucleic acid regions in the sample.

Efficiencies of amplification may vary between selected nucleic acid regions and between cycles so that in certain systems normalization (as described infra) may be used to ensure that the products from the amplification of the selected nucleic acid regions are representative of the nucleic acid content of the sample. One practicing the methods of the invention can mine the data regarding the relative frequency of the amplified products to determine variation in the selected nucleic acid regions, including variation in selected nucleic acid regions within a sample and/or between selected nucleic acid regions in different samples (particularly from the same selected nucleic acid regions in different samples) to normalize the data.

As an alternative to selective amplification, selected nucleic acid regions may be enriched by hybridization techniques (e.g., capture hybridization or hybridization to an array), optionally followed by one or more rounds of amplification. Optionally, the hybridized or captured selected nucleic acid regions are released (e.g., by denaturation) prior to amplification and sequence determination. The selected nucleic acid regions can be isolated from a maternal sample using various methods that allow for selective enrichment of the selected nucleic acid regions used in analysis. The isolation may be a removal of DNA in the maternal sample not used in analysis and/or removal of any excess oligonucleotides used in the initial enrichment or amplification step. For example, the selected nucleic acid regions can be isolated from the maternal sample using hybridization techniques, e.g., captured using binding of the selected nucleic acid regions to complementary oligos on a solid substrate such as a bead or an array, followed by removal of the non-bound nucleic acids from the sample. In another example, when a padlock-type probe technique is used for selective amplification (see, e.g., Barany et al., U.S. Pat. Nos. 6,858,412 and 7,556,924), the circularized nucleic acid products can be isolated from the linear nucleic acids, which are subject to selective degradation. Other useful methods of isolation will be apparent to one skilled in the art upon reading the present specification.

### Universal Amplification

The selectively-amplified copies of the selected nucleic acid regions may be amplified in a universal amplification step following the selective amplification or enrichment step, either prior to or during the detection step (i.e., sequencing or other detection technology). In performing universal amplification, universal primer sequences added to the copied selected nucleic acid region in the selective amplification step are used to further amplify the selected nucleic acid regions in a single universal amplification reaction. As described, universal primer sequences may be added to the copied selected nucleic acid regions during the selective amplification process, if performed, by using primers for the selective amplification step that have universal primer sequences so that the amplified copies of the selected nucleic acid regions incorporate the universal priming sequence. Alternatively, adapters comprising universal amplification sequences may be ligated to the ends of the selected nucleic acid regions following amplification or enrichment, if performed, and isolation of the selected nucleic acid regions from the maternal sample.

Bias and variability can be introduced into a sample during DNA amplification, and this is known to happen during polymerase chain reaction (PCR). In cases where an amplification reaction is multiplexed, there is the potential that selected nucleic acid regions will amplify at different rates or efficiencies, as each set of primers for a given selected nucleic acid region may behave differently based on the base composition of the primer and template DNA, buffer conditions, or other conditions. A universal DNA amplification for a multiplexed assay system generally introduces less bias and variability. Another technique to minimize amplification bias involves varying primer concentrations for different selected nucleic acid regions to limit the number of sequence specific amplification cycles in the selective amplification step. The same or different conditions (e.g., polymerase, buffers, and the like) may be used in the amplification steps, e.g., to ensure that bias and variability is not inadvertently introduced due to experimental conditions.

In a preferred aspect, a small number (e.g., 1-10, preferably 3-5) of cycles of selective amplification or nucleic acid enrichment is performed, followed by universal amplification using universal primers. The number of amplification cycles using universal primers will vary, but will preferably be at least 5 cycles, more preferably at least 10 cycles, even more preferably 20 cycles or more. By moving to universal amplification following one or a few selective amplification cycles, the bias of having certain selected nucleic acid regions amplify at greater rates than others is reduced.

Optionally, the assay system will include a step between the selective amplification and universal amplification to remove any excess nucleic acids that are not specifically amplified in the selective amplification. The whole product or an aliquot of the product from the selective amplification may be used for the universal amplification.

The universal regions of the primers used in the methods are designed to be compatible with conventional multiplexed methods that analyze large numbers of nucleic acids simultaneously in one reaction in one vessel. Such “universal” priming methods allow for efficient, high volume analysis of the quantity of nucleic acid regions present in a maternal sample, and allow for comprehensive quantification of the presence of nucleic acid regions within such a maternal sample for the determination of aneuploidy.

Examples of universal amplification methods include, but are not limited to, multiplexing methods used to amplify and/or genotype a variety of samples simultaneously, such as those described in Oliphant et al., U.S. Pat. No. 7,582,420, which is incorporated herein by reference.

In certain aspects, the assay system of the invention utilizes one of the following combined selective and universal amplification techniques: (1) the ligase detection reaction (“LDR”) coupled to polymerase chain reaction (“PCR”); (2) primary PCR coupled to secondary PCR coupled to LDR; and (3) primary PCR coupled to secondary PCR. Each of these combinations has particular utility for optimal detection. However, each of these combinations uses multiplex detection where oligonucleotide primers from an early phase of the assay system contains sequences that are utilized a later phase of the assay system.

Barany et al., U.S. Pat. Nos. 6,852,487, 6,797,470, 6,576,453, 6,534,293, 6,506,594, 6,312,892, 6,268,148, 6,054,564, 6,027,889, 5,830,711, 5,494,810, describe the use of the ligase chain reaction (LCR) assay for the detection of specific sequences of nucleotides in a variety of nucleic acid samples. Barany et al., U.S. Pat. Nos. 7,807,431, 7,455,965, 7,429,453, 7,364,858, 7,358,048, 7,332,285, 7,320,865, 7,312,039, 7,244,831, 7,198,894, 7,166,434, 7,097,980, 7,083,917, 7,014,994, 6,949,370, 6,852,487, 6,797,470, 6,576,453, 6,534,293, 6,506,594, 6,312,892, and 6,268,148 describe the use of LDR coupled with PCR for nucleic acid detection. Barany et al., U.S. Pat. Nos. 7,556,924 and 6,858,412, describe the use of padlock probes (also called “precircle probes” or “multi-inversion probes”) with coupled LDR and PCR for nucleic acid detection. Barany et al., U.S. Pat. Nos. 7,807,431, 7,709,201, and 7,198,814 describe the use of combined endonuclease cleavage and ligation reactions for the detection of nucleic acid sequences. Willis et al., U.S. Pat. Nos. 7,700,323 and 6,858,412, describe the use of precircle probes in multiplexed nucleic acid amplification, detection and genotyping. Ronaghi et al., U.S. Pat. No. 7,622,281 describes amplification techniques for labeling and amplifying a nucleic acid using an adapter comprising a unique primer and a barcode. Exemplary processes useful for amplifying and/or detecting selected nucleic acid regions include but are not limited to the methods described herein, each of which are incorporated by reference in their entirety for purposes of teaching various elements that can be used in the methods of the invention.

In addition to the various amplification techniques, numerous methods of sequence determination are compatible with the methods of the inventions. Preferably, such methods include “next generation” methods of sequencing. Exemplary methods for sequence determination include, but are not limited to, including, but not limited to, hybridization-based methods, such as disclosed in Drmanac, U.S. Pat. Nos. 6,864,052; 6,309,824; 6,401,267 and U.S. Pub. No. 2005/0191656, all of which are incorporated by reference; sequencing by synthesis methods, e.g., Nyren et al, U.S. Pat. Nos. 7,648,824, 7,459,311 and 6,210,891; Balasubramanian, U.S. Pat. Nos. 7,232,656 and 6,833,246; Quake, U.S. Pat. No. 6,911,345; Li et al, PNAS, 100: 414-19 (2003); pyrophosphate sequencing as described in Ronaghi et al., U.S. Pat. Nos. 7,648,824; 7,459,311; 6,828,100 and 6,210,891; and ligation-based sequencing determination methods, e.g., Drmanac et al., U.S. Pub. No. 2010/0105052, and Church et al, U.S. Pub. Nos. 2007/0207482 and 2009/0018024.

Alternatively, selected nucleic acid regions can be selected and/or identified using hybridization techniques. Methods for conducting polynucleotide hybridization assays for detection of have been well developed in the art. Hybridization assay procedures and conditions will vary depending on the application and are selected in accordance with the general binding methods known including those referred to in: Maniatis et al., Molecular Cloning: A Laboratory Manual (2nd Ed. Cold Spring Harbor, N.Y., 1989); Berger and Kimmel, Methods in Enzymology, Vol. 152, Guide to Molecular Cloning Techniques (Academic Press, Inc., San Diego, Calif., 1987); and Young and Davis, PNAS, 80:1194 (1983). Methods and apparatus for carrying out repeated and controlled hybridization reactions have been described in, e.g., U.S. Pat. Nos. 5,871,928; 5,874,219; 6,045,996; 6,386,749 and 6,391,623.

The present invention also contemplates signal detection of hybridization between ligands in certain preferred aspects. See U.S. Pat. Nos. 5,143,854; 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 60/364,731 and in PCT Application PCT/US99/06097 (published as WO99/47964).

Methods and apparatus for signal detection and processing of intensity data are disclosed in, for example, U.S. Pat. Nos. 5,143,854; 5,547,839; 5,578,832; 5,631,734; 5,800,992; 5,834,758; 5,856,092; 5,902,723; 5,936,324; 5,981,956; 6,025,601; 6,090,555; 6,141,096; 6,185,030; 6,201,639; 6,218,803 and 6,225,625, in U.S. Ser. No. 60/364,731 and in PCT Application PCT/US99/06097 (published as WO99/47964).

### Use of Indices in the Methods of the Invention

All or a portion of the selected nucleic acid regions may be directly detected using the described techniques, e.g., sequence determination or hybridization. However, in certain aspects the selected nucleic acid regions are associated with one or more indexes or indices that, e.g., identify the selected nucleic acid regions and/or a particular sample being analyzed. The detection of the one or more indices can serve as a surrogate for detection of the entire selected nucleic acid region, or detection of an index may serve as confirmation of the presence of a particular selected nucleic acid region if both the sequence of the index and the sequence of the nucleic acid region itself are determined. Indices are preferably associated with the selected nucleic acid regions during the selective amplification step using primers that comprise both the index and a region that specifically hybridizes to the selected nucleic acid region.

Indices are typically non-complementary, unique sequences used within an amplification primer to provide information relevant to the selected nucleic acid region that is isolated and/or amplified using the primer. In preferred aspects of the invention using indices, selective amplification primers are designed so that the one or more indices are coded in the primer. The order and placement of indices, as well as the length of indices, can vary, and they can be used in various combinations. Alternatively, the indices and/or universal amplification sequences can be added to the selectively-amplified selected nucleic acid regions following initial selective amplification using ligation of adaptors comprising these sequences. The advantage of employing indices is that the presence (and ultimately the quantity or frequency) of the selected nucleic acid regions can be obtained without the need to sequence the selected nucleic acid regions, although in certain aspects it may be desirable to do so. Generally, however, the ability to identify and quantify a selected nucleic acid region through identification of one or more indices will decrease the length of sequencing required, particularly if the index sequence is captured at the 3′ or 5′ end of the isolated selected nucleic acid region proximal to where a sequencing primer may be located. Use of indices as a surrogate for identification of selected nucleic acid regions also may reduce sequencing errors since longer sequencing reads are more prone to the introduction or error.

In one example of an index, the primers used for selective amplification of the selected nucleic acid regions are designed to include a locus index between the region complementary to the selected nucleic acid regions and the universal amplification primer site. A locus index typically is unique for each selected nucleic acid region so that quantification of the number of times a particular locus index occurs in a sample can be related to the relative number of copies of the corresponding single nucleic acid region and the particular chromosome containing the single nucleic acid region. Generally, the locus index is long enough to label each known single nucleic acid region uniquely. For instance, if the method uses 192 known single nucleic acid regions, there are at least 192 unique locus indexes, each uniquely identifying a single nucleic acid region from a particular locus on a chromosome. The locus indices used in the methods of the invention may be indicative of different single nucleic acid regions on a single chromosome as well as known single nucleic acid regions present on different chromosomes within a sample. The locus index may contain additional nucleotides that allow for identification and correction of sequencing errors including the detection of deletion, substitution, or insertion of one or more bases during sequencing as well as nucleotide changes that may occur outside of sequencing such as oligo synthesis, amplification, or any other aspect of the methods.

In another example, the primers used for amplification of the selected nucleic acid regions may be designed to provide an allele index (as an alternative to a locus index) between the region complementary to the selected nucleic acid region and the universal amplification primer site. An allele index is unique for a particular allele of a selected nucleic acid region, so that quantification of the number of times a particular allele index occurs in a sample can be related to the relative number of copies of that allele, and the summation of the allelic indices for a particular selected nucleic acid region can be related to the relative number of copies of that selected nucleic acid region on the particular chromosome containing the selected nucleic acid region.

In yet another example, the primers used for amplification of the selected nucleic acid regions may be designed to provide an identification index between the region complementary to a selected nucleic acid region and the universal amplification primer site. In such an aspect, a sufficient number of identification indices are present to uniquely identify each amplified molecule in the sample. Identification index sequences are preferably 6 or more nucleotides in length. In a preferred aspect, the identification index is long enough to have statistical probability of labeling each molecule with a single nucleic acid region uniquely. For example, if there are 3000 copies of a particular single nucleic acid region, there are substantially more than 3000 identification indexes such that each copy of a particular single nucleic acid region is likely to be labeled with a unique identification index. As with other indices, the identification index may contain additional nucleotides that allow for identification and correction of sequencing errors including the detection of deletion, substitution, or insertion of one or more bases during sequencing as well as nucleotide changes that may occur outside of sequencing such as oligo synthesis, amplification, and any other aspect of the assay.

The identification index may be combined with any other index to create one index that provides information for two properties. The identification locus may also be used to detect and quantify amplification bias that occurs downstream of the initial isolation of the selected nucleic acid regions from a sample and this data may be used to normalize the sample data.

In addition to the other indices described herein, a correction index may be employed. A correction index is a short nucleotide sequence that allows for correction of amplification, sequencing or other experimental errors including the detection of a deletion, substitution, or insertion of one or more bases during sequencing as well as nucleotide changes that may occur outside of sequencing such as oligonucleotide synthesis, amplification, or a other aspects of the assay. Correction indices may be stand-alone indices that are separate sequences, or they may be embedded within other indices to assist in confirming accuracy of the experimental techniques used, e.g., a correction index may be a subset of sequences of a locus index or an identification index.

In some aspects, indices that indicate the sample from which the selected nucleic acid regions are isolated are used to identify the source of the selected nucleic acid regions in a multiplexed assay system. In such aspects, the selected nucleic acid regions from one individual will be assigned to and associated with a particular unique sample index. The sample index can thus be used to assist in nucleic acid region identification for multiplexing of different samples in a single reaction vessel, such that each sample can be identified based on its sample index. In a preferred aspect, there is a unique sample index for each sample in a set of samples, and the samples are pooled during sequencing. For example, if twelve samples are pooled into a single sequencing reaction, there are at least twelve unique sample indexes such that each sample is labeled uniquely. After the sequencing step is performed, the sequencing data preferably is first segregated by sample index prior to determining the frequency of each the selected nucleic acid region for each sample and prior to determining whether there is a chromosomal abnormality for each sample.

### Detecting Fetal Chromosomal Aneuploidies

The present invention provides methods for identifying fetal Robertsonian translocations in maternal or paternal samples comprising nucleic acids or directly in samples comprising fetal nucleic acids. In certain embodiments, the sample are maternal sample comprising both maternal and fetal DNA such as maternal blood samples (i.e., whole blood, serum or plasma). The methods enrich and/or isolate one or, preferably, more selected nucleic acid regions in a maternal sample that correspond to individual chromosomes of interest and, in certain aspects, to reference chromosomes that are used to determine the presence or absence of a Robertsonian translocation. As described in detail supra, the methods of the invention preferably employ one or more selective amplification cycles (e.g., using one or more primers that specifically hybridize to the one or more selected nucleic acid regions) or enrichment (e.g., hybridization and separation) steps to enhance the content of the selected nucleic acid regions in the sample. The selective amplification and/or enrichment steps also preferably provide mechanisms to engineer the copies of the selected nucleic acid regions for further isolation, amplification or analysis. This is in direct contrast to the random amplification approach used by other techniques, e.g., massively parallel shotgun sequencing, as such techniques generally involve random amplification of all or a substantial portion of the genome.

In a general aspect, the user of the invention analyzes selected nucleic acid regions on different chromosomes simultaneously and in a preferred embodiment, all of the selected nucleic acid regions for each sample are amplified in one reaction vessel. In some embodiments, the selected nucleic acid regions from multiple samples are amplified in one reaction vessel, and the sample of origin of the different amplification products can be determined by use of a sample index.

One challenge with the detection of Robertsonian translocations in a fetus in a maternal sample is that the majority of the cell free fetal DNA as a percentage of total cell free DNA in a maternal sample such as blood serum or plasma may vary from less than one to forty percent, and most commonly is present at or below twenty percent and frequently at or below ten percent. In detecting a Robertsonian translocation, the relative increase of the extra q arm is 50% in the fetal DNA; thus, as a percentage of the total DNA in a maternal sample where, as an example, the fetal DNA is 10% of the total, the increase in the extra chromosome as a percentage of the total is 5%. The same principles apply when looking for a 10% decrease in the quantity or frequency of acrocentric conserved sequences. If one is to detect these differences robustly through the methods described herein, the variation in the measurement of the extra chromosome has to be significantly less than the percent increase of the extra chromosome. In some aspects where fetal contribution is high, a correction based upon fetal percent can be factored into the algorithm for detecting the Robertsonian translocation in the mother.

In preferred aspects, selected nucleic acid regions corresponding to multiple loci on a first chromosome are detected and summed to determine the relative frequency of a chromosome in the maternal sample. Next, selected nucleic acid regions corresponding to multiple loci on a second chromosome are detected and summed to determine the relative frequency of a chromosome in the maternal sample. Frequencies that are higher than expected for one chromosome when compared to the other chromosome in the maternal sample are indicative of a fetal duplication or aneuploidy from, e.g., the q arm of an acrocentric chromosome due to a Robertsonian translocation. When looking at p arm sequences (such as acrocentric conserved sequences) from acrocentric chromosomes, frequencies that are lower than expected when compared to another (control) chromosome in the maternal sample are indicative of loss of a p arm due to a Robertsonian translocation. The comparison may be between chromosomes that each may be a putative aneuploid in the fetus (e.g., chromosomes 13, 14, 15, 21 and 22), where the likelihood of both being aneuploid is minimal. The comparison can also be between chromosomes where one is putatively aneuploid (e.g., chromosome 13) and the other is very unlikely to be aneupolid (e.g., an autosome such as chromosome 12), which can act as a reference chromosome. In yet other aspects, the comparison may utilize two or more chromosomes that are putatively aneuploid (i.e., two or more chromosomes selected from chromosomes 13, 14, 15, 18, 21 and 22) and one or more reference chromosomes.

In one aspect, the assay system of the invention analyzes multiple selected nucleic acid regions representing selected loci on at least two chromosomes, and the relative frequency of each selected nucleic acid region from the sample is analyzed to determine a relative chromosome frequency for each chromosome. The chromosomal frequency of the at least two chromosomes is then compared to determine statistically whether a chromosomal abnormality exists.

In another aspect, the assay system of the invention analyzes multiple selected nucleic acid regions representing selected loci on chromosomes of interest, and the relative frequency of each selected nucleic acid region from the sample is analyzed and independently quantified to determine a relative frequency for each selected nucleic acid region in the sample. The sums of the selected nucleic acid regions in the sample are compared to statistically determine whether a chromosomal aneuploidy exists.

In another aspect, subsets of selected nucleic acid regions on each chromosome are analyzed to determine whether a chromosomal abnormality exists. The selected nucleic acid region frequency can be summed for a particular chromosome, and the summations of the selected nucleic acid regions used to determine anr aneuploidy. This aspect of the invention sums the frequencies of the individual selected nucleic acid regions from each chromosome and then compares the sum of the selected nucleic acid regions on one chromosome against another chromosome to determine whether a chromosomal abnormality exists. The subsets of selected nucleic acid regions can be chosen randomly but with sufficient numbers to yield a statistically significant result in determining whether a chromosomal abnormality exists. Multiple analyses of different subsets of selected nucleic acid regions can be performed within a maternal sample to yield more statistical power. For example, if there are 100 selected nucleic acid regions for chromosome 13 and 100 selected nucleic acid regions for chromosome 21, a series of analyses could be performed that evaluate fewer than 100 regions for each of the chromosomes. In another aspect, particular selected nucleic acid regions can be selected on each chromosome that are known to have less variation between samples, or by limiting the data used for determination of chromosomal frequency, e.g., by ignoring the data from selected nucleic acid regions with very high or very low frequency within a sample.

In a particular aspect, the ratio of the frequencies of the selected nucleic acid regions are compared to a reference mean ratio that has been determined for a statistically significant population of genetically “normal” subjects, i.e., subjects that do not have a Robertsonian translocation.

In a particular aspect, the measured quantity of one or more selected nucleic acid regions on a chromosome is normalized to account for known variation from sources such as the assay system (e.g., temperature, reagent lot differences), underlying biology of the sample (e.g., nucleic acid content), operator differences, or any other variables.

The data used to determine the frequency of the selected nucleic acid regions may exclude outlier data that appear to be due to experimental error, or that have elevated or depressed levels based on an idiopathic genetic bias within a particular sample. In one example, the data used for summation may exclude nucleic acid regions with a particularly elevated frequency in one or more samples. In another example, the data used for summation may exclude selected nucleic acid regions that are found in a particularly low abundance in one or more samples.

The quantity of different selected nucleic acid regions detectable on certain chromosomes may vary depending upon a number of factors, including general representation of fetal loci in maternal samples, degradation rates of the different nucleic acids representing fetal loci in maternal samples, sample preparation methods, and the like. Thus, in some aspects of the invention the frequencies of the individual selected nucleic acid regions on each chromosome are summed and then the sum of the selected nucleic acid regions on one chromosome are compared to the sum of an equal number of selected nucleic acid regions on another chromosome to determine whether a chromosomal abnormality exists.

The variation between samples and/or for selected nucleic acid regions within a sample may be minimized using a combination of analytical methods, many of which are described in this application. For instance, variation is lessened by using an internal reference in the assay. An example of an internal reference is the use of a chromosome present in a “normal” abundance (e.g., disomy for an autosome) to compare against the chromosome that may be present in abnormal abundance, i.e., the aneuploidy, in the same sample. While the use of a single such “normal” chromosome as a reference chromosome may be sufficient, it is also possible to use many normal chromosomes as the internal reference chromosomes to increase the statistical power of the quantification.

One utilization of an internal reference is to calculate a ratio of abundance of the putatively abnormal chromosomes or sub-chromosomal regions to the abundance of the normal chromosomes or sub-chromosomal regions in a sample, called a chromosomal ratio. In calculating the chromosomal ratio, the abundance or counts of each of the selected nucleic acid regions for each chromosome or sub-chromosomal region are summed together to calculate the total counts for each chromosome. The total counts for one chromosome are then divided by the total counts for a different chromosome or sub-chromosomal region to create a chromosomal ratio for those two chromosomes or sub-chromosomal regions.

Alternatively, a chromosomal ratio for each chromosome or sub-chromosomal region may be calculated by first summing the counts of each of the selected nucleic acid regions for each chromosome or sub-chromosomal region, and then dividing the sum for one chromosome or sub-chromosomal region by the total sum for two or more chromosomes. Once calculated, the chromosomal ratio is then compared to the average chromosomal ratio from a normal population.

The average may be the mean, median, mode or other average, with or without normalization and exclusion of outlier data. In a preferred aspect, the mean is used. In developing the data set for the chromosomal ratio from the normal population, the normal variation of the measured chromosomes or sub-chromosomal regions is calculated. This variation may be expressed a number of ways, most typically as the coefficient of variation, or CV. When the chromosomal ratio from the sample is compared to the average chromosomal ratio from a normal population, if the chromosomal ratio for the sample falls statistically outside of the average chromosomal ratio for the normal population, the sample contains an aneuploidy (Robertsonian translocation). The criteria for setting the statistical threshold to declare an aneuploidy depend upon the variation in the measurement of the chromosomal ratio and the acceptable false positive and false negative rates for the desired assay. In general, this threshold may be a multiple of the variation observed in the chromosomal ratio. In one example, this threshold is three or more times the variation of the chromosomal ratio. In another example, it is four or more times the variation of the chromosomal ratio. In another example it is five or more times the variation of the chromosomal ratio. In another example it is six or more times the variation of the chromosomal ratio. In the example above, the chromosomal ratio is determined by summing the counts of selected nucleic acid regions by chromosome or sub-chromosomal region. Typically, the same number of selected nucleic acid regions for each chromosome or sub-chromosomal region is used. An alternative method for generating the chromosomal ratio would be to calculate the average counts for the selected nucleic acid regions for each chromosome or chromosomal region. The average may be any estimate of the mean, median or mode, although typically an average is used. The average may be the mean of all counts or some variation such as a trimmed or weighted average. Once the average counts for each chromosome or sub-chromosomal region have been calculated, the average counts for each chromosome or sub-chromosomal region may be divided by the other to obtain a chromosomal ratio between two chromosomes, the average counts for each chromosome may be divided by the sum of the averages for all measured chromosomes to obtain a chromosomal ratio for each chromosome as described above. As highlighted above, the ability to detect a Robertsonian translocation in a maternal sample where the putative DNA is in low relative abundance depends greatly on the variation in the measurements of different selected nucleic acid regions in the assay. Numerous analytical methods can be used that reduce this variation and thus improve the sensitivity of this method to detect aneuploidy.

One method for reducing variability of the assay is to increase the number of selected nucleic acid regions used to calculate the abundance of the chromosomes or sub-chromosomal regions. In general, if the measured variation of a single selected nucleic acid region of a chromosome is X % and Y different selected nucleic acid regions are measured on the same chromosome, the variation of the measurement of the chromosomal abundance calculated by summing or averaging the abundance of each selected nucleic acid region on that chromosome will be approximately X % divided by Y1/2. Stated differently, the variation of the measurement of the chromosome abundance would be approximately the average variation of the measurement of each selected nucleic acid region's abundance divided by the square root of the number of selected nucleic acid regions.

In a preferred aspect of this invention, the number of selected nucleic acid regions measured for each chromosome is at least 10. In another preferred aspect of this invention the number of selected nucleic acid regions measured for each chromosome is at least 24. In yet another preferred aspect of this invention, the number of selected nucleic acid regions measured for each chromosome is at least 48. In another preferred aspect of this invention, the number of selected nucleic acid regions measured for each chromosome is at least 100. In another preferred aspect of this invention the number of selected nucleic acid regions measured for each chromosome is at least 200. There is incremental cost to measuring each selected nucleic acid region and thus it is important to minimize the number of each selected nucleic acid region while still generating statistically robust data. In a preferred aspect of this invention, the number of selected nucleic acid regions measured for each chromosome is less than 2000. In a preferred aspect of this invention, the number of selected nucleic acid regions measured for each chromosome is less than 1000. In a most preferred aspect of this invention, the number of selected nucleic acid regions measured for each chromosome is at least 48 and less than 1000. In one aspect, following the measurement of abundance for each selected nucleic acid region, a subset of the selected nucleic acid regions may be used to determine the presence or absence of a Roberstonian translocation. There are many standard methods for choosing the subset of selected nucleic acid regions. These methods include outlier exclusion, where the selected nucleic acid regions with detected levels below and/or above a certain percentile are discarded from the analysis. In one aspect, the percentile may be the lowest and highest 5% as measured by abundance. In another aspect, the percentile may be the lowest and highest 10% as measured by abundance. In another aspect, the percentile may be the lowest and highest 25% as measured by abundance.

Another method for choosing a subset of selected nucleic acid regions include the elimination of regions that fall outside of some statistical limit. For instance, regions that fall outside of one or more standard deviations of the mean abundance may be removed from the analysis. Another method for choosing the subset of selected nucleic acid regions may be to compare the relative abundance of a selected nucleic acid region to the expected abundance of the same selected nucleic acid region in a healthy population and discard any selected nucleic acid regions that fail the expectation test. To further minimize the variation in the assay, the number of times each selected nucleic acid region is measured may be increased. As discussed, in contrast to the random methods of detecting Robertsonian translocations and other aneuploidies where the genome is measured on average less than once, the methods of the present invention intentionally measures each selected nucleic acid region multiple times. In general, when counting events, the variation in the counting is determined by Poisson statistics, and the counting variation is typically equal to one divided by the square root of the number of counts. In a preferred aspect of the invention, the selected nucleic acid regions are each measured on average at least 100 times. In a preferred aspect to the invention, the selected nucleic acid regions are each measured on average at least 500 times. In a preferred aspect to the invention, the selected nucleic acid regions are each measured on average at least 1000 times. In a preferred aspect to the invention, the selected nucleic acid regions are each measured on average at least 2000 times. In a preferred aspect to the invention, the selected nucleic acid regions are each measured on average at least 5000 times.

In another aspect, subsets of selected nucleic acid regions can be chosen randomly using sufficient numbers to yield a statistically significant result in determining whether a chromosomal abnormality exists. Multiple analyses of different subsets of selected nucleic acid regions can be performed within a maternal sample to yield more statistical power. In this example, it may or may not be necessary to remove or eliminate any selected nucleic acid regions prior to the random analysis. For example, if there are 100 selected nucleic acid regions for chromosome 13 and 100 selected nucleic acid regions for chromosome 14, a series of analyses could be performed that evaluate fewer than 100 regions for each of the chromosomes.

Sequence counts also can be normalized by systematically removing sample and assay biases by using median polish on log-transformed counts. A metric can be computed for each sample as the means of counts for a selected nucleic acid region divided by the sum of the mean of counts for selected nucleic acid regions on a particular chromosome and the mean of courts for the selected nucleic acid regions on a different chromosome. A standard Z test of proportions may be used to compute Z statistics:

$${Z}_{j} = \frac{{p}_{j} - {p}_{0}}{\sqrt{\frac{{p}_{j} ( 1 - {p}_{j} )}{{n}_{j}}}}$$

where pj is the observed proportion for a given chromosome of interest in a given sample j, p0 is the expected proportion for the given test chromosome calculated as the median pj, and nj is the denominator of the proportion metric. Z statistic standardization may be performed using iterative censoring. At each iteration, the samples falling outside of, e.g., three median absolute deviations are removed. After ten iterations, mean and standard deviation were calculated using only the uncensored samples. All samples are then standardized against this mean and standard deviation. The Kolmogorov-Smirnov test (see Conover, Practical Nonparametric Statistics, pp. 295-301 (John Wiley & Sons, New York, N.Y., 1971)) and Shapiro-Wilk's test (see Royston, Applied Statistics, 31:115-124 (1982)) may be used to test for the normality of the normal samples' Z statistics.

In addition to the methods above for reducing variation in the assay, other analytical techniques, many of which are described earlier in this application, may be used in combination. For example, the variation in the assay may be reduced when all of the selected nucleic acid regions for each sample are interrogated in a single reaction in a single vessel. Similarly, the variation in the assay may be reduced when a universal amplification system is used. Furthermore, the variation of the assay may be reduced when the number of cycles of amplification is limited.

### Determination of Fetal DNA Content in Maternal Sample

In certain specific aspects and as described herein, determining the percentage of fetal DNA in a maternal sample may increase the accuracy of the frequency calculations for the selected nucleic acid regions, as knowledge of the fetal contribution provides important information on the expected statistical presence of the selected nucleic acid regions. Variation from the expectation may be indicative of chromosome copy number variation associated with Robertsonian translocations. Taking percent fetal into account may be particularly helpful in circumstances where the level of fetal DNA in a maternal sample is low, as the percent fetal contribution can be used to determine the quantitative statistical significance in the variations of levels of selected nucleic acid regions in a maternal sample.

In some specific aspects, the relative maternal contribution of maternal DNA at an allele of interest can be compared to the non-maternal contribution at that allele to determine approximate fetal DNA concentration in the sample. In other specific aspects, the relative quantity of solely paternally-derived sequences (e.g., Y-chromosome sequences or paternally-specific polymorphisms) can be used to determine the relative concentration of fetal DNA in a maternal sample. Another exemplary approach to determining the percent fetal contribution in a maternal sample is through the analysis of DNA fragments with different patterns of DNA methylation between fetal and maternal DNA.

In circumstances where the fetus is male, percent fetal DNA in a sample can be determined through detection of Y-specific nucleic acids and comparison to calculated maternal DNA content. Quantities of an amplified Y-specific nucleic acid, such as a region from the sex-determining region Y gene (SRY), which is located on the Y chromosome and is thus representative of fetal DNA, can be determined from the sample and compared to one or more amplified genes which are present in both maternal DNA and fetal DNA and which are preferably not from a chromosome believed to potentially be aneuploid in the fetus, e.g., an autosomal region that is not on chromosome 13, 14, 15, 18, 21, or 22. Preferably, this amplification step is performed in parallel with the selective amplification step, although it may be performed either before or after the selective amplification depending on the nature of the multiplexed assay.

In particular aspects, the percentage of cell free fetal DNA in the maternal sample can determined by PCR using serially diluted DNA isolated from the maternal sample, which can accurately quantify the number of genomes comprising the amplified genes. For example, if the blood sample contains 100% male fetal DNA, and 1:2 serial dilutions are performed, then on average the SRY signal will disappear 1 dilution before the autosomal signal, since there is 1 copy of the SRY gene and 2 copies of the autosomal gene.

In a specific aspect, the percentage of free fetal DNA in maternal plasma is calculated using the following formula: percentage of free fetal DNA=(No. of copies of SRY gene×2×100)/(No. of copies of autosomal gene), where the number of copies of each gene is determined by observing the highest serial dilution in which the gene was detected. The formula contains a multiplication factor of 2, which is used to normalize for the fact that there is only 1 copy of the SRY gene compared to two copies of the autosomal gene in each genome, fetal or maternal.

In some circumstances such as with a female fetus, the determination of fetal polymorphisms requires targeted SNP and/or mutation analysis to identify the presence of fetal DNA in a maternal sample. In some aspects, the use of prior genotyping of the father and mother can be performed. For example, the parents may have undergone such genotype determination for identification of disease markers, e.g., determination of the genotype for disorders such as cystic fibrosis, muscular dystrophy, spinal muscular atrophy or even the status of the RhD gene may be determined. Such difference in polymorphisms, copy number variants or mutations can be used to determine the percentage fetal contribution in a maternal sample.

In an alternative preferred aspect, the percent fetal cell free DNA in a maternal sample can be quantified using multiplexed SNP detection without using prior knowledge of the maternal or paternal genotype. In this aspect, two or more selected polymorphic nucleic acid regions with a known SNP in each region are used. In a preferred aspect, the selected polymorphic nucleic acid regions are located on an autosomal chromosome that is unlikely to be aneuploid, e.g., Chromosome 6. The selected polymorphic nucleic acid regions from the maternal sample are amplified where the amplification is universal.

In a preferred embodiment, the selected polymorphic nucleic acid regions are amplified in one reaction in one vessel. Each allele of the selected polymorphic nucleic acid regions in the maternal sample is determined and quantified using, e.g., high throughput sequencing. Following sequence determination, loci are identified where the maternal and fetal genotypes are different, e.g., the maternal genotype is homozygous and the fetal genotype is heterozygous. This identification is accomplished by observing a high relative frequency of one allele (>60%) and a low relative frequency (<20% and >0.15%) of the other allele for a particular selected nucleic acid region. The use of multiple loci is particularly advantageous as it reduces the amount of variation in the measurement of the abundance of the alleles. All or a subset of the loci that meet this requirement are used to determine fetal concentration through statistical analysis.

In one aspect, fetal concentration is determined by summing the low frequency alleles from two or more loci together, dividing by the sum of the high and low frequency alleles and multiplying by two. In another aspect, the percent fetal cell free DNA is determined by averaging the low frequency alleles from two or more loci, dividing by the average of the high and low frequency alleles and multiplying by two.

For many alleles, maternal and fetal sequences may be homozygous and identical, and as this information does not distinguish between maternal and fetal DNA, it is not useful in the determination of percent fetal DNA in a maternal sample. The present invention utilizes allelic information where there is a difference between the fetal and maternal DNA (e.g., a fetal allele containing at least one allele that differs from the maternal allele) in calculations of percent fetal. Data pertaining to allelic regions that are the same for the maternal and fetal DNA are thus not selected for analysis, or are removed from the pertinent data prior to determination of percentage fetal DNA so as not to swamp out the useful data. Exemplary methods for quantifying fetal DNA in maternal plasma can be found, e.g., in Chu et al., Prenat Diagn, 30:1226-29 (2010), which is incorporated herein by reference.

In one aspect, selected nucleic acid regions may be excluded if the amount or frequency of the region appears to be an outlier due to experimental error, or from idiopathic genetic bias within a particular sample. In another aspect, selected nucleic acids may undergo statistical or mathematical adjustment such as normalization, standardization, clustering, or transformation prior to summation or averaging. In another aspect, selected nucleic acids may undergo both normalization and data experimental error exclusion prior to summation or averaging. In a preferred aspect, 12 or more loci are used for the analysis. In another preferred aspect, 24 or more loci are used for the analysis. In another preferred aspect, 48 or more loci are used for the analysis. In another aspect, one or more indices are used to identify the sample, the locus, the allele or the identification of the nucleic acid.

In one preferred aspect, the percentage fetal contribution in a maternal sample can be quantified using tandem SNP detection in the maternal and fetal alleles. Techniques for identifying tandem SNPs in DNA extracted from a maternal sample are disclosed in Mitchell et al, U.S. Pat. No. 7,799,531 and U.S.SNs. 12/581,070; 12/581,083; 12/689,924 and 12/850,588. These references describe the differentiation of fetal and maternal loci through detection of at least one tandem single nucleotide polymorphism (SNP) in a maternal sample that has a different haplotype between the fetal and maternal genome. Identification and quantification of these haplotypes can be performed directly on the maternal sample, as described in the Mitchell et al. disclosures, and used to determine the percent fetal contribution in the maternal sample.

In yet another alternative, certain genes have been identified as having epigenetic differences between the maternal and fetal gene copies, and such genes are candidate loci for fetal DNA markers in a maternal sample. See, e.g., Chim, et al., PNAS USA, 102:14753-58 (2005). These loci, which may be methylated in the fetal DNA but unmethylated in maternal DNA (or vice versa), can be readily detected with high specificity by use of methylation-specific PCR (MSP) even when such fetal DNA molecules were present among an excess of background plasma DNA of maternal origin. The comparison of methylated and unmethylated amplification products in a maternal sample can be used to quantify the percent fetal DNA contribution to the maternal sample by calculating the epigenetic allelic ratio for one or more of such sequences known to be differentially regulated by methylation in the fetal DNA as compared to maternal DNA.

To determine methylation status of nucleic acids in a maternal sample, the nucleic acids of the sample are subjected to bisulfite conversion of the samples and then subjected to MSP, followed by allele-specific primer extension. Conventional methods for such bisulphite conversion include, but are not limited to, use of commercially available kits such as the Methylamp™ DNA Modification Kit (Epigentek, Brooklyn, N.Y.). Allelic frequencies and ratios can be directly calculated and exported from the data to determine the relative percentage of fetal DNA in the maternal sample.

### Use of Percent Fetal Cell Free DNA to Optimize Fetal Aneuploidy Detection

Once percent fetal cell free DNA has been calculated, this data may be combined with methods for aneuploidy detection to determine the likelihood that a fetus may contain an aneuploidy such as a Robertsonian translocation. In one aspect, an aneuploidy detection method that utilizes analysis of random DNA segments is used, such as that described in, e.g., Quake, U.S. Ser. No. 11/701,686; and Shoemaker et al., U.S.SN No. 12/230,628. In a preferred aspect, aneuploidy detection methods that utilize analysis of selected nucleic acid regions are used. In this aspect, the percent fetal cell free DNA for a sample is calculated. The chromosomal ratio for that sample, a chromosomal ratio for the normal population and a variation for the chromosomal ratio for the normal population is determined, as described herein.

In one preferred aspect, the chromosomal ratio and its variation for the normal population are determined from normal samples that have a similar percentage of fetal DNA. An expected aneuploid chromosomal ratio for a DNA sample with that percent fetal cell free DNA is calculated by adding the percent contribution from the aneuploid chromosome. The chromosomal ratio for the sample may then be compared to the chromosomal ratio for the normal population and to the expected aneuploid chromosomal ratio to determine statistically, using the variation of the chromosomal ratio, if the sample is more likely normal or aneuploid, and the statistical probability that it is one or the other.

In a preferred aspect, the selected regions of a maternal sample include both regions for determination of fetal DNA content as well as non-polymorphic regions from two or more chromosomes to detect a Roberstonian translocation in a single reaction. The single reaction helps to minimize the risk of contamination or bias that may be introduced during various steps in the assay system which may otherwise skew results when utilizing fetal DNA content to help determine the presence or absence of a chromosomal abnormality.

In other aspects, a selected nucleic acid region or regions may be utilized both for determination of fetal DNA content as well as detection of fetal chromosomal abnormalities. The alleles for selected nucleic acid regions can be used to determine fetal DNA content and these same selected nucleic acid regions can then be used to detect fetal chromosomal abnormalities ignoring the allelic information. Utilizing the same selected nucleic acid regions for both fetal DNA content and detection of chromosomal abnormalities may further help minimize any bias due to experimental error or contamination.

In one embodiment, fetal source contribution in a maternal sample regardless of fetal gender is measured using autosomal SNPs (see, Sparks, et al., Am. J. Obstet & Gyn., 206:319.e1-9 (2012)). The processes utilized do not require prior knowledge of paternal genotype, as the non-maternal alleles are identified during the methods without regard to knowledge of paternal inheritance. A maximum likelihood estimate using the binomial distribution may be used to calculate the estimated fetal nucleic acid contribution across several informative loci in each maternal sample. The processes for calculation of fetal acid contribution used are described, for example, in U.S. Ser. No. 61/509,188 (Atty Docket No. ARIA007PRV), which is incorporated by reference. The polymorphic regions used for determination of fetal contribution may be from chromosomes 1-12, and preferably do not target the blood group antigens. The estimate of fetal contribution from the polymorphic assays is used to define expected response magnitudes when a test chromosome is trisomic, which informs the statistical testing. The test statistic may consist of two components: a measure of deviation from the expected proportion when the sample is disomic; and a measure of deviation from the expected proportion when the sample is trisomic. Each component is in the form of a Wald statistic (e.g., Harrell, Regression modeling strategies, (2001, Springer-Verlag), Sections 9.2.2 and 10.5) which compares an observed proportion to an expected proportion and divides by the variation of the observation.

The statistic Wj may be used to measure the deviation from expectation when the sample j is disomic, and is defined as

$${W}_{j} = \frac{{p}_{j} - {p}_{0}}{{σ}_{{p}_{j}}} ,$$

where pj and p0 are defined as described supra with the Z statistic, and σₚ_{ⱼ} is the standard deviation of the observed proportion of representation for a given chromosome of interest. The standard deviation may be estimated using parametric bootstrap sampling to create a distribution of pj proportions based on the mean counts and standard errors for our chromosomes of interest. The second statistic is Ŵⱼ, which replaces p0 with the fetal fraction adjusted reference proportion \hat{p}ⱼ is defined as

$${\hat{p}}_{j} = \frac{( 1 + 0.5 {f}_{j} ) {p}_{0}}{( ( 1 + 0.5 {f}_{j} ) {p}_{0} ) ( 1 - {p}_{0} )} ,$$

where fⱼ is the fetal fraction for sample j and p₀ is the reference proportion as before. This adjustment accounts for the increased representation of a test chromosome when the fetus was trisomic. Because this variance of counts across many loci is measured as a natural result of using multiple non-polymorphic assays for ther test chromosomes, all estimates are taken within a nascent data set and do not require external reference samples or historical information with normalizing adjustments to control for process drift as is typically required for variance around the expected proportion.

The final statistic used was Sⱼ=Wⱼ+Ŵⱼ. Conceptually, deviations from disomic expectation and trisomic expectation are simultaneously evaluated and summarized into this single statistic. The particular advantage of combining these two indicators is that while deviation from disomy might be high, it may not reach the deviation expected for trisomy at a particular fetal contribution level. The Ŵⱼ component will be negative in this case, in effect penalizing the deviation from disomy. An Sⱼ=0 indicated an equal chance of being disomic vs. trisomic.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1

### Detection of Robertsonian Translocations Using Fixed Sequence Oligonucleotides

In a first embodiment, assays directed against specific genomic regions were used to identify the presence or absence of a Robertsonian translocation involving those chromosomes. The present assay system allowed the identification of the presence or absence of such a loss in the DNA of multiple individuals using a highly multiplexed system.

Multiple interrogations were prepared using oligonucleotides complementary to or derived from regions of interest on chromosomes 13, 14, 15, 21 and/or 22. Each separate assay interrogation consisted of two fixed sequence oligos that hybridize to genomic regions of interest in chromosomes 13, 14, 15, 21 and/or 22. The fixed sequence oligonucleotides used may vary depending on whether the assay is interrogating specific selected regions on the chromosomes, or interrogating regions of the p arms that are conserved between chromosomes.

The first oligos, complementary to the 3′ region, comprised the following sequential (5′ to 3′) elements: a universal PCR priming sequence common to all assays: TACACCGGCGTTATGCGTCGAGAC (SEQ ID NO:1); a nine nucleotide identification code specific to the 3′ region; a hybridization breaking nucleotide different from the corresponding base in the region; and a 20-24 bp sequence complementary to the genomic region. These first oligos were designed to provide a predicted uniform Tₘ with a 1.1 degree variation across all interrogations in the 8 assay set.

The second fixed sequence oligo, complementary to the 5′ region, comprised the following sequential (5′ to 3′) elements: a 20-24 bp sequence complimentary to the 5′ region; a hybridization breaking nucleotide which was different from the corresponding base in the region; and a universal PCR priming sequence which was common to all third oligos in the assay set:

(SEQ ID NO: 2)
ATTGCGGGGACCGATGATCGCGTC.

No polymorphic assays were used. In certain tested aspects, one or more bridging oligos were used that were complementary to the region between the region complementary to the first and second fixed sequence oligos. The length of the bridging oligonucleotides used in the assay systems varied from 5 to 50 base pairs.

All oligonucleotides used in the tandem ligation formats were synthesized using conventional solid-phase chemistry. The oligos of the first fixed set and the bridging oligonucleotides were synthesized with 5′ phosphate moieties to enable ligation to 3′ hydroxyl termini of adjacent oligonucleotides.

### Example 2

### Preparation of DNA for Use in Tandem Ligation Procedures

A total of 560 genomic DNA samples from both males and females, were fragmented by acoustic shearing (Covaris, Woburn, Mass.) to a mean fragment size of approximately 200 bp.

The DNA was biotinylated using standard procedures. Briefly, the Covaris fragmented DNA was end-repaired by generating the following reaction in a 1.5 ml microtube: 50 ng/μl DNA, 10 μl 10× T4 ligase buffer (Enzymatics, Beverly Mass.), 10 U T4 polynucleotide kinase (Enzymatics, Beverly Mass.), and H20 to 480 μl. This was incubated at 37° C. for 30 minutes. The DNA was diluted using 1000 mM Tris, 500 mM EDTA, pH 8.0, 10% Tween-80, 1000 ng/μl Yeast RNA Carrier Stock, and H2O to desired final concentration of ˜0.5 ng/μl.

DNA was placed in each well of a 96-well plate, 72 μl of 0.6× AM1 was dispensed into each well, and the plate was vortexed at 1200 rpm for 5 minutes. The plate was incubated at room temperature for 5 minutes and incubated on a post magnet for 10 minutes. 192 μl supernatant from the plate was transferred to each well of a new 96-deep well plate containing 384 μl of AM2. The new plate was vortexed at 1200 rpm for five minutes and incubated for 5 minutes at room temperature. The plate was then incubated on a post magnet for 20 minutes and the supernatant was discarded. 200 μl of 70% ethanol was added to each well of the 96-deep well plate and incubated for five minutes on the post magnet. The supernatant was discarded. This washing procedure was repeated once. The plate was then incubated on the magnet for 5 minutes at room temperature. 25 μl of a solution containing 1000 mM Tris, 500 mM EDTA, pH 8.0, 10% Tween-80, and H₂O was added to each well and the plate was vortexed at 1200 rpm for 5 minutes. The plate was then incubated on a post magnet for 2 minutes and the supernatant was transferred to a fresh 96-well PCR plate. The plate was sealed with an adhesive plate sealer and incubated at 95° C. for 3 minutes, and cooled to 10° C., and spun again for 10 seconds at 250×g. A biotinylation master mix was prepared in a 1.5 ml microtube to final concentration of: 10×“Green Buffer” (Enzymatics, Beverly Mass.), 20 U/μl TdT (Enzymatics, Beverly Mass.), 10.0% Tween-80, 1.0 nmol/μl biotin-16-dUTP (Roche, Nutley N.J.), and H₂0 to 1.5 ml. 7.5 μl of the master mix was aliquoted into each well of a 96-well plate. The plate was vortexed at 1200 rpm for 1 minute and the plate was sealed with an adhesive plate sealer. The plate was then incubated for 37° C. for 60 minutes and cooled to 10° C. Following incubation, the plate was spun for 10 seconds at 250×g, and 11.25 μl precipitation mix (1 ng/μl Dextran Blue, 3M NaOAC) was added to each well.

The plate was vortexed for 1 minute at 1800 rpm. 41.25 μl of isopropanol was added into each well, the plate sealed with adhesive plate sealer, and vortexed for 1 minute at 1900 rpm. The plate was spun for 20 minutes at 3000×g, the supernatant was decanted, and the plate inverted and centrifuged at 10×g for 1 minute onto an absorbent wipe. The pellet was resuspended in 30 μl solution containing 1000 mM Tris, 500 mM EDTA, pH 8.0, 10% Tween-80, 1000 ng/μl and H2O and vortexed for 3 minutes at 1900 rpm. An equimolar pool (40 nM each) of sets of first and second loci-specific fixed oligonucleotides was created from the oligos prepared as set forth above. A separate equimolar pool (20 μM each) of bridging oligonucleotides was likewise created for the assay processes based on the sequences of the selected genomic loci.

5 mg of strepavidin beads were transferred into a single 15 ml conical tube on a 15 ml magnetic stand and the supernatant was discarded. 6 ml binding buffer (1000 mM Tris pH 8.0, 500 mM EDTA, 5000 mM NaCl₂, 100% formamide, 10% Tween-80) was added to the ml conical tube and the beads were resuspended by vortexing, and 1 ml 40 nM fixed sequence oligo pool was added. 70 μl of this solution was added to each well of the 96-well plate prepared in Example 2 and the plate was vortexed for 1 minute at 1200 rpm. The plate was sealed with an adhesive plate sealer and the oligos were annealed to the template DNA by incubation at 70° C. for 5 minutes, followed by slow cooling to 30° C. and the plate was spun for 10 seconds at 250×g.

The plate was placed on a raised bar magnetic plate for 2 minutes to pull the magnetic beads and associated DNA to the side of the wells. The supernatant was removed by pipetting, and was replaced with 50 μL of 60% binding buffer (v/v in water). The beads were resuspended by vortexing, placed on the magnet again, and the supernatant was removed. This bead wash procedure was repeated once using 50 uL 60% binding buffer, and repeated twice more using 50 μL wash buffer (1000 mM Tris pH 8.0, 500 mM EDTA, 5000 mM NaCl₂, 10.0% Tween-80, H₂O).

The beads were resuspended in 37 μl ligation reaction mix containing 10× Taq ligase buffer (Enzymatics, Beverly Mass.), 40U Taq ligase, 10.0% Tween-80, H20, and 100 uM bridging oligo pool, and vortexed for 1 minute at 1900 rpm. The plate was sealed with an adhesive plate sealer, and incubated at 45° C. for one hour, cooled to 10° C. and spun for 10 seconds at 250×g. The plate was placed on a raised bar magnetic plate for 2 minutes to pull the magnetic beads and associated DNA to the side of the wells. The supernatant was removed by pipetting, and was replaced with 50 μL wash buffer. The beads were resuspended by vortexing, placed on the magnet again, and the supernatant was removed. The wash procedure was repeated once.

To elute the products from the strepavidin beads, 30 μl of 1000 mM Tris, 500 mM EDTA, pH 8.0, 10% Tween-80, 1000 ng/μl Yeast RNA Carrier Stock, and H2O was added to each well of 96-well plate. The plate was sealed and vortexed for 1 minute at 1900 rpm to resuspend the beads. The plate was incubated at 95° C. for 1 minute, cooled to 10° C., and spun for 10 seconds at 250×g. The plate seal was removed and placed on a raised-bar magnet plate for two minutes and the supernatant aspirated using an 8-channel pipetter. 25 μl of supernatant from each well was transferred into a fresh 96-well plate for universal amplification.

### Example 3

### Universal Amplification of Ligated Products

The polymerized and/or ligated nucleic acids were amplified using universal PCR primers complementary to the universal sequences present in the first and second fixed sequence oligos hybridized to the nucleic acid regions of interest. 25 μl of each of the reaction mixtures of Example 3 were used in each amplification reaction. A 50 μL universal PCR reaction consisting of 25 μL eluted ligation product plus 5× Phusion buffer (Finnzymes, Finland), 5M Betaine, 25 mM each dNTP, 2 U Phusion High-Fidelity DNA polymerase, 10.0% Tween-80, and the following primer pairs:

(SEQ ID NO: 3)
TAATGATACGGCGACCACCGAGATCTACACCGGCGTTATGCGTCGAGA
and
(SEQ ID NO: 4)
TCAAGCAGAAGACGGCATACGAGATXAAACGACGCGATCATCGGTCCCC
GCAA,

where X represents one of 96 different sample tags used to uniquely identify individual samples prior to pooling and sequencing. The plate was sealed with an adhesive plate sealer and PCR was carried out under stringent conditions using a BioRad Tetrad™ thermocycler. The plate was then spun for 10 seconds at 250×g.

10 μl of universal PCR product from each of the samples were pooled and 100 μl was dispensed into 8 separate tubes of a new 96-deep well plate and 100 μl of AM1 was added to each. The tubes were vortexed for 1 minute at 1200 rpm and incubated at room temperature for 5 minutes. The plate was placed on a Post Magnet for 5 minutes and the supernatant was discarded. 200 μl of 70% Ethanol was added into each well and the plate was incubated at room temperature for 30 seconds. The supernatant was decanted and discarded. This wash procedure was repeated once and the place was removed from the Post Magnet. 25 μl of a solution containing 1000 mM Tris, 500 mM EDTA, pH 8.0, 10% Tween-80, and H₂O was dispensed into each ell and the mixture was vortexed for 1 minute at 1200 rpm. The mixture was incubated for 1 minute at room temperature and the plate was placed on a Post Magnet for 2 minutes. 25 μL from each well were pooled in a 1.5 ml tube. The pooled PCR product was purified using AMPure™ SPRI beads (Beckman-Coulter, Danvers, Mass.), and quantified using Quant-iT™ PicoGreen, (Invitrogen, Carlsbad, Calif.).

### Example 4

### Interrogation of Selected Regions on Chromosome 14

Multiple fixed sequence interrogations were prepared using oligonucleotides complementary to or derived from FISH probe pTRS63 (a region on the p arm of chromosome 14), which has been previously shown to specifically hybridize to the p arm of chromosome 14 (Choo, et al., Am. J. Hum. Genet., 50:706-16 (1992)). Eight separate assay interrogations were performed, each consisting of two fixed sequence oligos that hybridize in the PTRS63 genomic region (the selected nucleic acid region). The first oligos, complementary to the 3′ region, comprised the following sequential (5′ to 3′) elements: a universal PCR priming sequence common to all assays: TACACCGGCGTTATGCGTCGAGAC (SEQ ID NO:1); a nine nucleotide identification code specific to the 3′ region; a hybridization breaking nucleotide different from the corresponding base in the PTRS63 region; and a 20-24 bp sequence complementary to the pTRS63 genomic region. These first oligos were designed to provide a predicted uniform Tm with a 1.1 degree variation across all interrogations in the 8 assay set.

The second fixed sequence oligo, complementary to the 5′ region of the PTRS63 region, comprised the following sequential (5′ to 3′) elements: a 20-24 bp sequence complimentary to the 5′ region of the pTRS63 region; a hybridization breaking nucleotide which was different from the corresponding base in the PTRS63 region; and a universal PCR priming sequence which was common to all third oligos in the assay set:

(SEQ ID NO: 2)
ATTGCGGGGACCGATGATCGCGTC.

The interrogations were carried out using the methods as described in Examples 1-3. The purified PCR products of were sequenced on a single lane of a slide on an IIlumina HiSeq 2000. Sequencing runs typically give rise to ˜100M raw reads, of which ˜85M (85%) map to expected assay structures. This translated to an average of ˜885K reads/sample across the experiment, and (in the case of an experiment using 96 loci) 9.2K reads/replicate/locus across 96 selected nucleic acid regions.

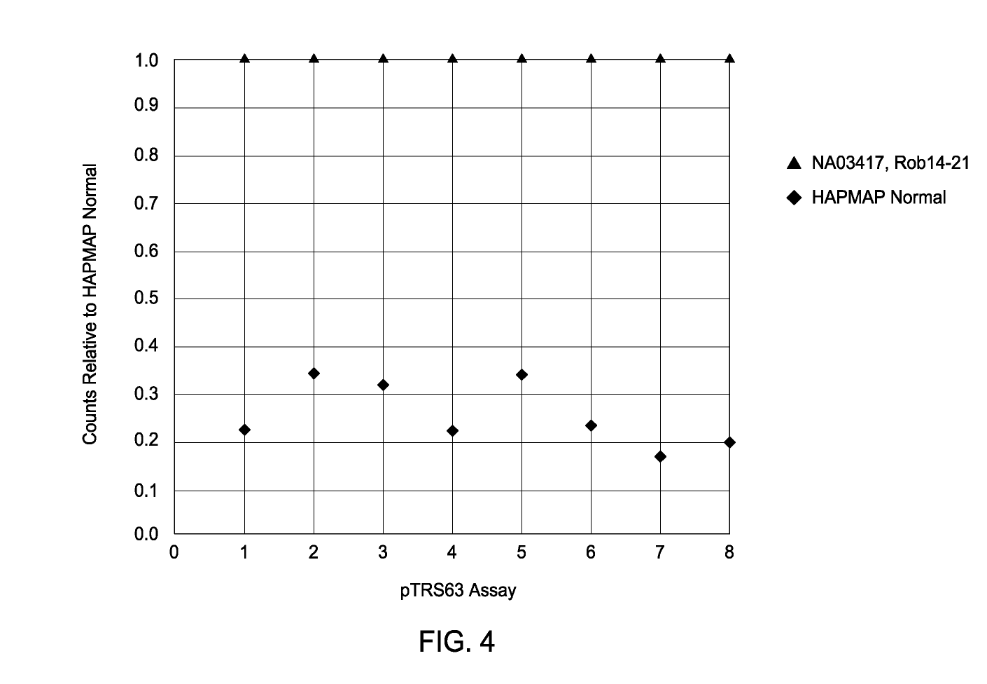

FIG. 4 is a graph comparing the frequency of the pTRS63 sequence from the p arm of chromosome 14 from a control sample and in a sample with a known 14-21 Robertsonian translocation. Note that the control is set to 1.0, and the relative number of counts for the pTRS63 sequence in the Rob 14-21 averages around 0.25.

### Example 5

### Interrogation of Regions on Chromosomes 13, 14, 15 21, and 22

Multiple fixed sequence interrogations were prepared using oligonucleotides complementary to or derived from regions on the p arm that are conserved between chromosomes 13, 14, 15, 21, and/or 22. Assay interrogations were performed, each consisting of two fixed sequence oligos that hybridize in regions of the p arm of chromosomes 13, 14, 15, 21 and/or 22 (selected regions). The first oligos, complementary to the 3′ region, comprised the following sequential (5′ to 3′) elements: a universal PCR priming sequence common to all assays: TACACCGGCGTTATGCGTCGAGAC (SEQ ID NO:1); a nine nucleotide identification code specific to the 3′ region; a hybridization breaking nucleotide different from the corresponding base in the selected regions; and a 20-24 bp sequence complementary to the selected regions. These first oligos were designed to provide a predicted uniform Tm with a 1.1 degree variation across all interrogations in the 8 assay set.

The second fixed sequence oligo, complementary to the 5′ region of the selected regions, comprised the following sequential (5′ to 3′) elements: a 20-24 bp sequence complimentary to the 5′ region of the selected regions; a hybridization breaking nucleotide which was different from the corresponding base in the selected regions; and a universal PCR priming sequence which was common to all third oligos in the assay set:

(SEQ ID NO: 2)
ATTGCGGGGACCGATGATCGCGTC.

The interrogations were carried out using the methods as described in Examples 1-3.

The purified PCR products of were sequenced on a single lane of a slide on an IIlumina HiSeq 2000. Sequencing runs typically give rise to ˜100M raw reads, of which ˜85M (85%) map to expected assay structures. This translated to an average of ˜85K reads/sample across the experiment, and (in the case of an experiment using 96 loci) 9.2K reads/replicate/locus across 96 selected nucleic acid regions.

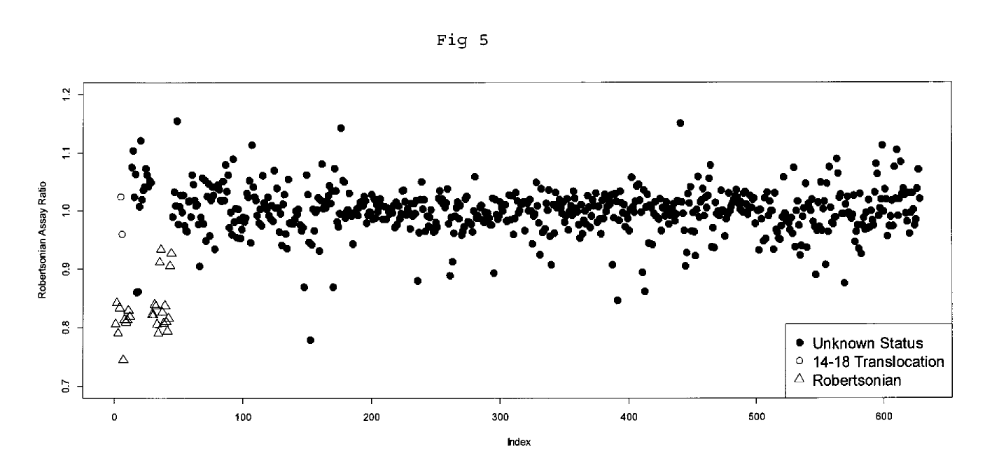

FIG. 5 is a graph showing the Robertsonian Assay Ratio of each of the 560 samples. Note, samples known to comprise a Robertsonian translocation were found to have a Robertsonian Assay Ratio below 1.0, as would be expected.

While this invention is satisfied by aspects in many different forms, as described in detail in connection with preferred aspects of the invention, it is understood that the present disclosure is to be considered as exemplary of the principles of the invention and is not intended to limit the invention to the specific aspects illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. §112, §16.

1. A method for detecting the presence or absence of a Robertsonian translocation in an individual comprising the steps of:
providing DNA samples from at least five individuals;
selectively amplifying one or more selected nucleic acid regions from the p arm of a first chromosome in the samples, wherein the first chromosome is selected from chromosome 13, 14, 15, 21 or 22, and wherein the primers used for selective amplification comprise universal amplification regions;
selectively amplifying one or more selected nucleic acid regions from a region outside the p arm of the first chromosome in the samples, wherein the primers used for selective amplification comprise universal amplification regions;
further amplifying the selected nucleic acid regions from the at least five individual samples in a single universal amplification reaction;
detecting the amplified nucleic acid regions resulting from the single amplification reaction; and
calculating a relative frequency of the selected nucleic acid regions for an individual sample;
comparing the relative frequencies of the selected nucleic acid regions from the p arm of the first chromosome and the selected nucleic acid regions from outside the p arm of the first chromosome for an individual sample; and
identifying the presence or absence of a Robertsonian translocation in an individual sample based on the compared relative frequencies.

2. The method of claim 1, wherein the DNA samples are from maternal samples.

3. The method of claim 1, wherein the selected nucleic acid region from the p arm of the first chromosome is acrocentric conserved sequence.

4. The method of claim 3, where a relative frequency of the selected nucleic acid region from the p arm of the first chromosome in an individual that is approximately 10% lower than the relative frequency for the one or more selected nucleic acid regions outside the p arm of the first chromosome in the individual is indicative of a Robertsonian translocation in the individual.

5. The method of claim 1, wherein at least ten selected nucleic acid regions from the p arm of the first chromosome are amplified.

6. The method of claim 5, wherein at least forty-eight selected nucleic acid regions from the p arm of the first chromosome are amplified.

7. The method of claim 6, wherein at least ninety-six selected nucleic acid regions from the p arm of the first chromosome are amplified.

8. The method of claim 1, wherein the selected nucleic acid region from the p arm of the first chromosome is a chromosome-specific genomic region.

9. The method of claim 8, wherein a relative frequency of approximately 50% for the selected nucleic acid region from the p arm of the first chromosome of an individual compared to the relative frequency of the one or more selected nucleic acid regions outside the p arm on the first chromosome in that individual is indicative of a Robertsonian translocation.

10. The method of claim 1, wherein at least one of the selected nucleic acid regions outside the p arm of the first chromosome is from the q arm of the first chromosome.

11. The method of claim 1, wherein at least one of the selected nucleic acid regions outside the p arm of the first chromosome is from a second chromosome.

12. The method of claim 11, wherein the second chromosome is selected from chromosome 1, 2, 3, 4, 5, 6, 7, 8, 9, 10, 11, 12, 16, 17, 18, 19, 20, or 23.

13. The method of claim 1, wherein the selected nucleic acid regions are associated with one or more identifying indices.

14. The method of claim 15, wherein the frequency of the selected nucleic acid regions is quantified through detection of the associated one or more indices.

15. The method of claim 1, where the DNA is selectively amplified in a single vessel.

16. The method of claim 1, where the selected nucleic acid regions are each counted an average of at least 250 times.

17. The method of claim 1, wherein the DNA samples are from at least twenty-four individuals.

18. The method of claim 19, wherein the DNA samples are from at least forty eight individuals.

19. The method of claim 21, wherein the DNA samples are from at least ninety-six individuals.

20. A method for detecting the presence or absence of a Robertsonian translocation in an individual comprising the steps of:
providing DNA samples from at least five individuals;
selectively amplifying one or more selected nucleic acid regions from the p arm of a first chromosome in the individual samples, wherein the first chromosome is selected from chromosome 13, 14, 15, 21 or 22;
selectively amplifying one or more selected nucleic acid regions from a region outside the p arm of the first chromosome in the individual samples;
introducing universal amplification sequences to the selectively amplified nucleic acids regions of the individual samples, each comprising a sample-specific index;
pooling the individual samples;
further amplifying the selected nucleic acid regions from the at least five individual samples in a single universal amplification reaction;
detecting the amplified nucleic acid regions resulting from the single amplification reaction; and
calculating a relative frequency of the selected nucleic acid regions for an individual sample;
comparing the relative frequencies of the selected nucleic acid regions from the p arm of the first chromosome and the selected nucleic acid regions outside the p arm of the first chromosome for an individual sample; and
identifying the presence or absence of a Robertsonian translocation in an individual sample based on the compared relative frequencies.

21. The method of claim 22, wherein the DNA sample is from a maternal sample.

22. The method of claim 22, wherein the selected nucleic acid region from the p arm of the first chromosome is an acrocentric conserved sequence.

23. The method of claim 22, wherein at least ten selected nucleic acid regions from the p arm of the first chromosome are amplified.

24. The method of claim 25, wherein at least forty-eight selected nucleic acid regions from the p arm of the first chromosome are amplified.

25. The method of claim 26, wherein at least ninety-six selected nucleic acid regions from the p arm of the first chromosome are amplified.

26. The method of claim 24, where a relative frequency of the selected nucleic acid region from the p arm of the first chromosome in an individual that is approximately 10% lower than the relative frequency for the one or more selected nucleic acid regions outside the p arm of the first chromosome in the individual is indicative of a Robertsonian translocation in the individual.

27. The method of claim 22, wherein the selected nucleic acid region from the p arm of the first chromosome is a chromosome-specific genomic region.

28. The method of claim 29, wherein at least two selected nucleic acid regions from the p arm of the first chromosome are amplified.

29. The method of claim 30, wherein at least ten selected nucleic acid regions from the p arm of the first chromosome are amplified.

30. The method of claim 29, wherein a relative frequency of the selected nucleic acid region from the p arm of the first chromosome in an individual is approximately 50% than the relative frequency for the one or more selected nucleic acid regions outside the p arm on the first chromosome in that individual is indicative of a Robertsonian translocation.

31. The method of claim 22, wherein at least one of the selected nucleic acid regions outside the p arm of the first chromosome is from the q arm of the first chromosome.

32. The method of claim 22, wherein at least one of the selected nucleic acid regions outside the p arm of the first chromosome is from a second chromosome.

33. The method of claim 22, wherein the at least second chromosome is selected from chromosome 1, 2, 3, 4, 5, 6, 7, 8, 9, 10, 11, 12, 16, 17, 18, 19, 20, or 23.

34. The method of claim 22, wherein the selected nucleic acid regions are associated with one or more identifying indices.

35. The method of claim 36, wherein the frequency of the selected nucleic acid regions is quantified through detection of the associated one or more indices.

36. The method of claim 22, where the selected nucleic acid regions are each counted an average of at least 250 times.


SEQUENCE LISTING (4 sequences)

SEQ ID NO: 1 - DNA, 24 bp, Artificial Sequence
  Universal primer sequence
tacaccggcgttatgcgtcgagac

SEQ ID NO: 2 - DNA, 24 bp, Artificial Sequence
  Universal primer sequence
attgcggggaccgatgatcgcgtc

SEQ ID NO: 3 - DNA, 48 bp, Artificial Sequence
  Universal primer sequence
taatgatacggcgaccaccgagatctacaccggcgttatgcgtcgaga

SEQ ID NO: 4 - DNA, 56 bp, Artificial Sequence
  Universal primer sequence
tcaagcagaagacggcatacgagatnnnnaaacgacgcgatcatcggtccccgcaa